SEQUENCE LISTING (3 sequences)

SEQ ID NO: 1 - DNA, 18 bp, Artificial Sequence
  forward primer
gaggagatcaggcccaag

SEQ ID NO: 2 - DNA, 20 bp, Artificial Sequence
  reverse primer
cgttcaccacgtcatagtag

SEQ ID NO: 3 - DNA, 26 bp, Artificial Sequence
  sequence after being ligated into vector
aagggcatcatcaccatcaccactga

(19) United States
(12) Patent Application Publication
(10) Pub. No.: US 2004/0265909 A1
(43) Pub. Date: Dec. 30, 2004

(54) Compound libraries and methods for drug discovery

(76) Inventors: Jeff Blaney, Piedmont, CA (US); Ian McDonald, San Diego, CA (US); Masaki Tomimoto, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/821,662

(22) Filed: Apr. 9, 2004

Related U.S. Application Data
(60) Provisional application No. 60/462,638, filed on Apr. 11, 2003.
(60) Provisional application No. 60/531,197, filed on Dec. 19, 2003.

(51) Int. Cl. G01N 33/53; G01N 33/543

(52) U.S. Cl. 435/7.1; 436/518

(57) ABSTRACT

The present invention provides compound libraries that can be rapidly and efficiently synthesized for use in lead discovery and design, and can be easily expanded for further drug discovery. The present invention also provides methods of using such compound libraries to design and identify novel drug leads.

This application claims priority to U.S. provisional applications by Blaney et al., entitled Compound Libraries and Methods for Drug Discovery, filed on Apr. 11, 2003, Provisional Ser. No. 60/462,638, and filed on Dec. 19, 2003, Provisional Ser. No. 60/531,197, which are both hereby incorporated by reference herein in their entirety. This application is also related to PCT Application No. PCT/US04/______, by Blaney, et al. and having the above title, filed Apr. 9, 2004 with attorney docket number 022132001110PC, which is hereby incorporated by reference herein in its entirety.

### INTRODUCTION

Novel compounds are continually sought after to treat and prevent diseases and disorders. The advent of combinatorial chemistry has allowed researchers to design, then synthesize, thousands to hundred thousands of compounds to use in the development of novel therapeutics. Pharmaceutical companies, and companies specializing in combinatorial chemistry, can purchase compound libraries containing great numbers of compounds or build facilities to synthesize such libraries, then screen using high throughput screening, thousands to millions of compounds for activity against a particular target. Using these methods, compounds are selected that have the most desirable results in assays, for example, those that have the strongest binding profile. This method of drug discovery is generally available only to those companies that have the resources to conduct this type of research. But, even for such companies, this method relies on a massive amount of effort and resources, and, in the end, relies on almost randomly finding a binding compound, or compound having biochemical activity, a “hit,” that can be developed into a lead compound. Moreover, the lead candidate is often too large for further development. It is often desirable to modify a lead candidate to improve its solubility, absorption, metabolic properties, or other properties. If a lead candidate is too large to be modified and still retain desirable therapeutic properties, then researchers lose valuable time.

The results obtained from high throughput screening of massive numbers of compounds are limited; researchers select the compounds that are randomly found to have the best assay results. These compounds may not necessarily be the ones that ultimately have the best chance of therapeutic success. In addition, hundreds to thousands of false positives may occur in the initial screens. These false positives may be due to any number of factors, including, but not limited to, the “noise” of the assay, aggregation, protein denaturing, and interference with the assay. Researchers must expend a significant amount of effort assaying these false positive compounds, as well as developing compounds that would not meet therapeutic criteria. Furthermore, many potential lead compounds can be overlooked because of less desirable initial assay results. These compounds having false negative results may indeed lead to valuable lead candidates, having desirable therapeutic qualities. But, under traditional screening methods, these compounds may likely be overlooked. Good leads, especially weakly active inhibitors, are often very hard to detect using traditional high throughput screening approaches.

Also, once a compound is selected for having desirable assay results, another round of combinatorial synthesis is often then performed, to optimize the lead compound. This optimization is often conducted in a blind fashion, in that random changes are then made to different parts of the lead molecule, which are then screened. This leads to more inefficiencies and lost opportunities.

One way that researchers have attempted to reduce the number of compounds needed for screening is to perform an initial computational screen, docking numerous compounds to protein models, to determine which compounds are most likely to bind to the target. The result of such efforts can often include compounds that are difficult to synthesize, which can severely limit the scope of actual screening, and the start-up time required for high throughput screening.

Thus, many problems exist using current combinatorial library-based drug discovery methods. First, a large amount of resources must be spent to conduct this type of research, second, computational methods designed to reduce the number of actual assays often select compounds that may be difficult to synthesize, also wasting time and resources, third, binding and biological activity assays may easily overlook lead candidates that do not meet a certain threshold of activity, and information about compounds that do have activity does not direct the scientist to the appropriate modifications to make to improve the compound's activity, and fourth lead candidates are often identified that are too large to use in developing improved properties without wasting research time.

The present invention provides a solution to these problems, providing a method of drug discovery that does not require the resources and time necessary to obtain huge compound libraries consisting of hundreds of thousands of compounds, one that allows researchers to recognize and optimize weak hits, one that increases the likelihood that the initial hits can be developed to meet therapeutic requirements such as, for example, ADMET requirements, and one that provides information to guide researchers in lead optimization.

Citation of documents herein is not intended as an admission that any is pertinent prior art. All statements as to the date or representation as to the contents of documents is based on the information available to the applicant and does not constitute any admission as to the correctness of the dates or contents of the documents.

### BRIEF DESCRIPTION OF THE FIGURES

FIG. 1 represents an example of the design of linear compound libraries.

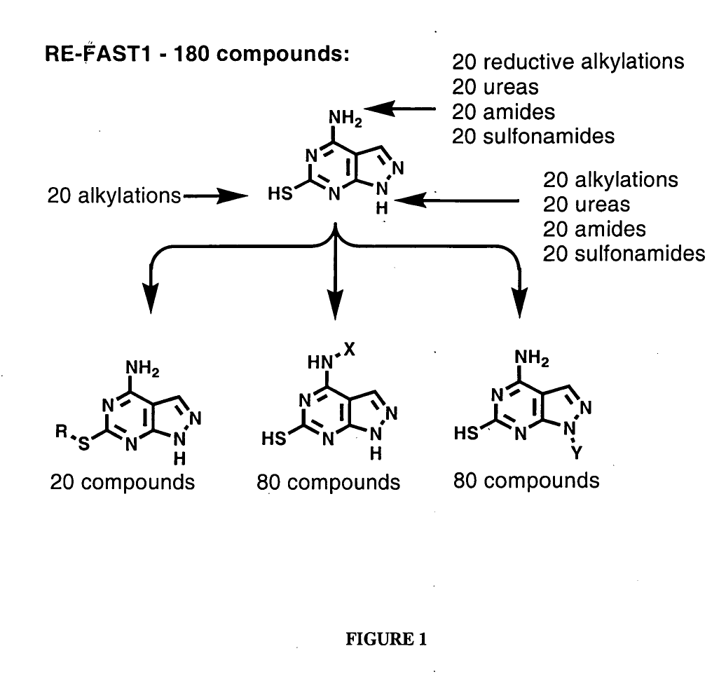

FIG. 2 depicts methods of using handles to generate derivative compound libraries.

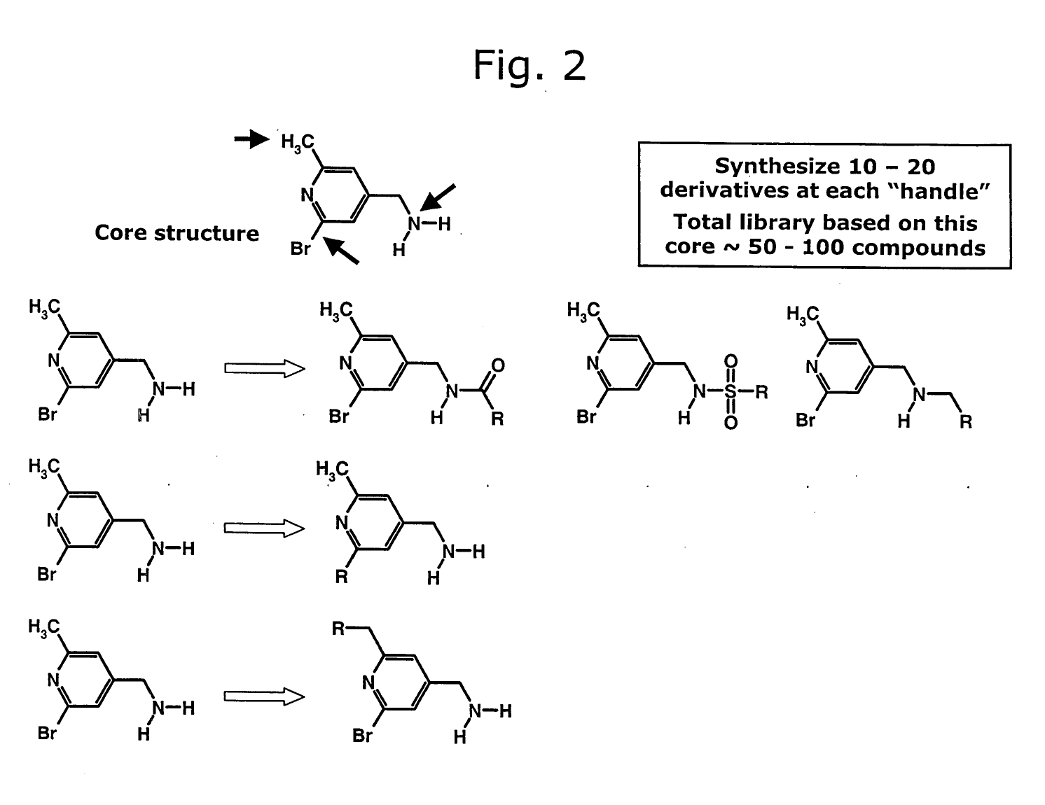

FIG. 3 depicts a method of inital screening of the present invention comprising using computational chemistry to assist in determining the appropriate handles in linear compound libraries, and incorporating in vitro assays to determine SAR.

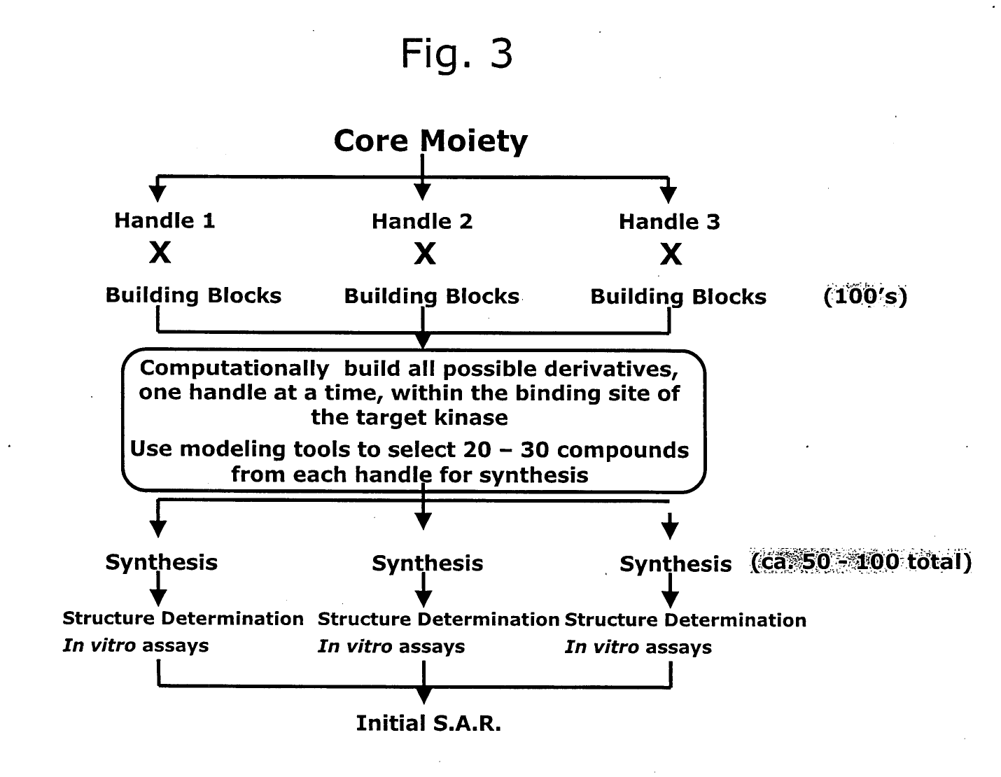

FIG. 4 depicts a method of optimization of the present invention comprising further developing an initial SAR using combinatorial libraries developed from compunds selected from the linear library, to obtain a more active compound.

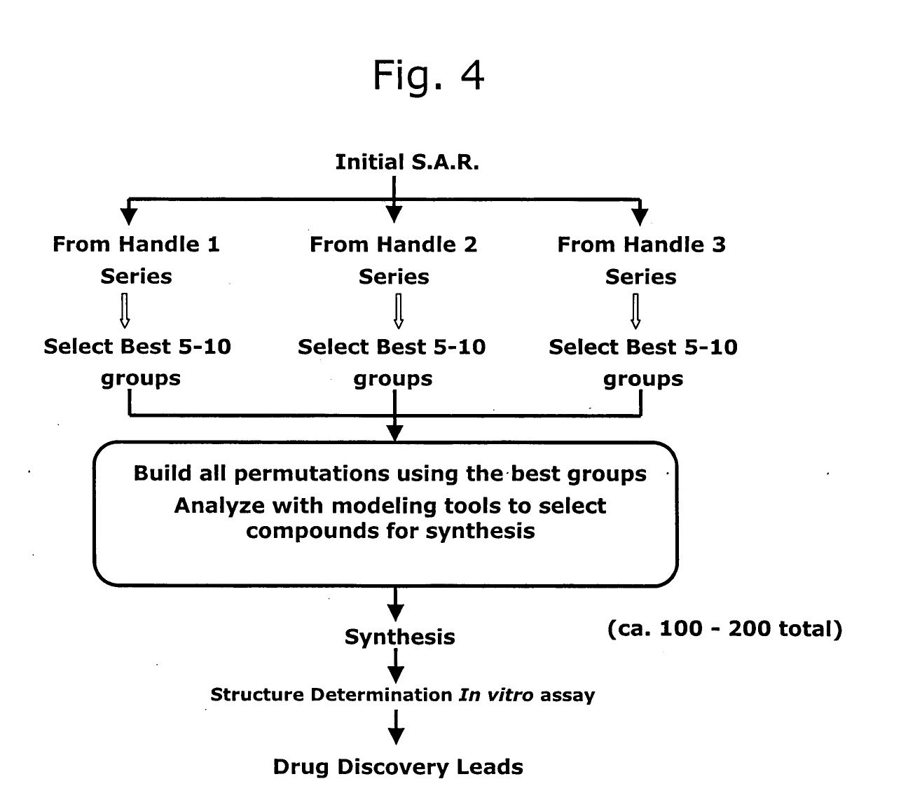

FIG. 5 represents an example of the design of a combinatorial library of FIG. 4.

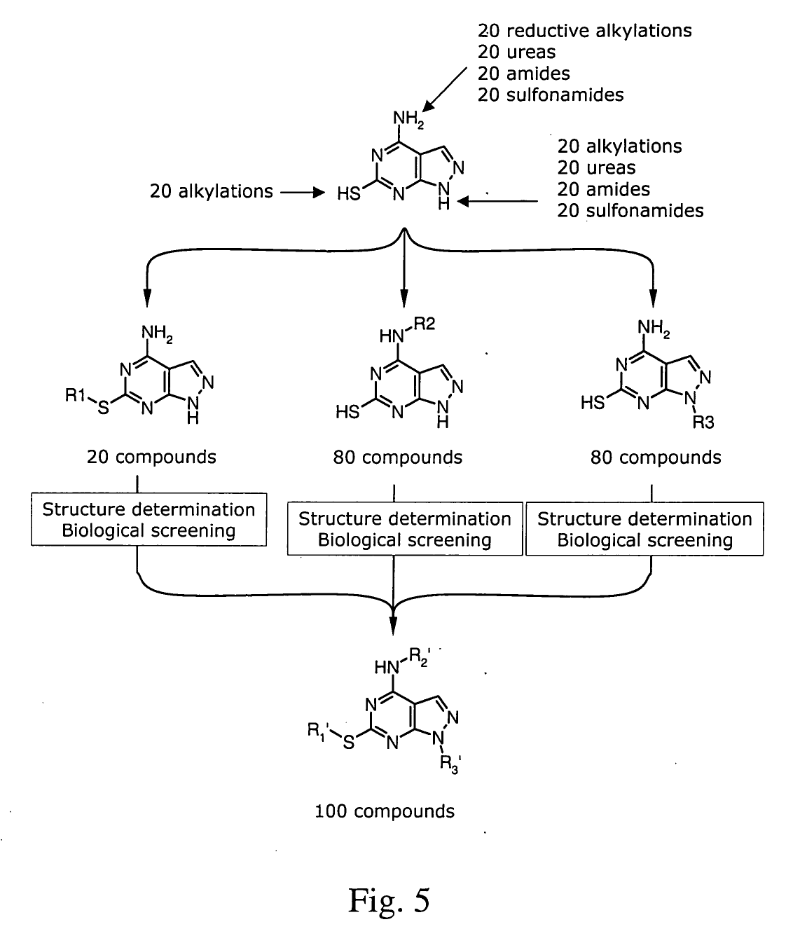

FIG. 6 depicts a method of computationally designing a linear library, by selecting central core/handle combinations.

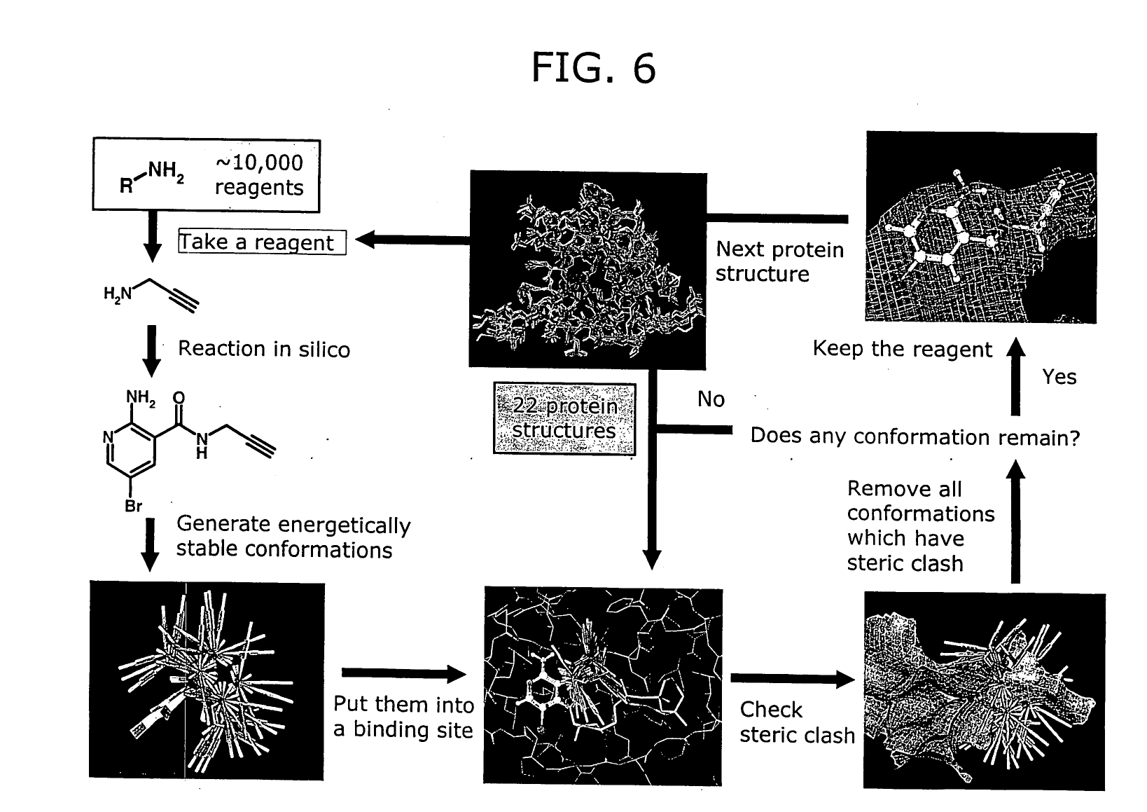

FIG. 7 depicts an example of the design of a kinase inhibitor using aspects of the methods of the present invention.

FIG. 8 provides a list of examples of synthetic methods that may be used in the one step and two step synthesis methods of the present invention. Synthetic methods may be found in, for example, March's Advanced Organic Chemistry: Reactions, Mechanisms, and Structure, 5th Edition (Michael B. Smith and Jerry March, Wiley Interscience 2001, ISBN 0-471-58589-0), hereby incorporated by reference herein in its entirety.

### DETAILED DESCRIPTION OF THE INVENTION

The present invention provides methods of designing core fragment libraries useful for developing lead compounds that have therapeutic qualities. The present invention also provides libraries that comprise core fragments that, in the same molecule, have visualization and functional properties, that is, they are designed to be easily used for structure determination and to be easily modified for drug design. The present invention integrates the use of biophysical or biochemical assays with the determination of the physical interaction between one or more test compounds, such as those of a library, and the biological target molecule. The present invention provides a method of rapidly and efficiently discovering lead compounds and generating information to guide optimization of the lead compounds for improved activity. In one aspect, the present invention incorporates the process of analyzing crystals of core fragments in association with a biological target molecule, using crystallization or soaking, to avoid problems including, but not limited to, those inherent in traditional high throughput screening. The present invention also provides a method of rapidly, efficiently, and systematically exploring a binding site of a target molecule to design compounds that optimally fit in that binding site, by providing a roadmap to optimize potency, selectivity, and drug-like properties.

In traditional high throughput screening, because of problems with the background “noise” of the assay, aggregation, denatured proteins, and assay interference, there may be hundreds to thousands of false weak hits, that is, compounds that show some activity, but not within the desired range or detection threshold or cutoff of the assay. The present invention allows for initial screening using limited selected core fragment libraries, followed by the ability to exploit “weak hits,” core fragments having lower than desired activity, for further drug discovery. Thus, a large combinatorial library is not required for initial discovery research. Instead, once a researcher has some direction from crystal visualization as to the general design of compounds useful for a particular target, a large, but more focused, combinatorial library may then be used.

Therapeutically desirable properties of drug candidates include those meeting certain criteria, such as, for example, ADMET, and the Lipinski rule of five. Using multiple relatively small core fragments or core moieties in the initial screening libraries has the advantage in that lead candidates can be designed using small core fragments as a base. Many of the core fragments comprise a substituent that has anomalous dispersion properties. For example, bromine (Br) atoms have anomalous dispersion properties that assist in X-ray crystallographic determination of the precise orientation of a fragment when it is bound to a biological target molecule. Bromine atoms also, for example, serve as a useful substituent for one or two step synthetic chemistry. These core fragments are used to obtain initial leads, and by modifying substituents or adding larger or smaller substituents the core fragments can be modified to be more potent. In addition, space left on the compounds may be used to add substituents that aid in meeting therapeutic requirements. For example, substituents that enhance solubility can be added without resulting in a compound that is too large to be developed into a therapeutically effective compound.

The present invention provides a core fragment library comprising a plurality of core fragments, wherein said core fragments have the formula

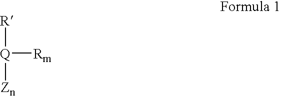

wherein

Z is a handle capable of anomalous dispersion;

Q is a central core;

Q may be the same or different on each compound;

Each R is, independently, H or a handle;

Each R′ is, independently, H or a handle;

n is an integer 0 or greater;

m is an integer 0 or greater; and

(m+n) cannot be greater than the number of available bonds on Q.

The term “majority” generally refers to half (50%) or greater than about half of a given population. In some embodiments of the invention, the term may be defined as greater than about 60%, greater than about 70%, greater than about 80%, or greater than about 90%. In one example, m=0, m may, for example, be less than six, less than five, less than four, less than three, or less than two. In one example, n=0, and m may, for example, be less than three, or less than two. In one example, n is 1, and m is two. In. some aspects of the invention, R′ is at the same position on Q for at least about 95%, about 90%, or about 75% of the compounds in the library. In some aspects of the invention, n=1 and for at least about 95%, about 90%, or about 75% of the compounds, each compound differs from each other compound only at R′. In some aspects of the invention, n is an integer 1 or grater, and Z is independently selected from the group consisting of Br, R″Br, S, SR″, Se, SeR′, Cl; and each R″ is, independently, H or a functional group, such as, for example, a straight or branched alkyl or heteroalkyl, an alkenyl, an alkynyl, a ring or a fused ring, functional groups may be modified with additional substituents. In some aspects of the present invention, ‘R’ is selected from the group consisting of the handles of Table 1. In some aspects of the present invention, Z is selected from the group consisting of the handles of Table 1 that comprise Br. In some aspects of the present invention, R″ is selected from the group consisting of the handles of Table 1, for example selected from a group of the handles of Table 1 that do not comprise Br. In one aspect of the present invention, Z is Br or R″Br. The present invention also provides a mixture comprising a biological target molecule and a core fragment library.

The present invention also provides a linear library, for example, in one aspect is provided a compound library comprising a plurality of compounds, wherein said compounds have the formula

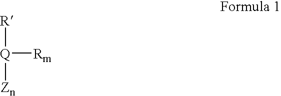

wherein

Z is a handle capable of anomalous dispersion;

Q is a central core, and for each compound, Q is the same;

Each R is, independently, H or a handle;

Each R′ is, independently, H or a derived substituent;

n is an integer 0 or greater; m is an integer 0 or greater; and

(m+n) cannot be greater than the number of available bonds on Q;

with the provisos that

for the majority of compounds in the library, the same R groups are at the same position on Q;

for the majority of compounds in the library, R′ is at the same position on Q; and for the majority of compounds in the library, each n is the same.

The present invention also provides a mixture comprising a biological target molecule and a compound of the linear library.

In one aspect of the invention, a core fragment library is provided comprising a plurality of core fragments wherein each of the core fragments comprises two or more handles, and less than 17 non-hydrogen atoms. The core fragment may comprise a central core comprising at least one single or fused ring system, or the core fragment may comprise a central core that does not include a closed ring. The core fragment may, for example, comprise at least one hetero atom, the at least one heteroatom may, for example, be part of a ring in the central core. In one aspect, the invention provides a core fragment library comprising a plurality of core fragments wherein each of the core fragments comprises two or more handles, at least about 50%, at least about 75%, at least about 90%, or at least about 95% of the core fragments have less than four hydrogen bond donors, at least about 50%, at least about 75%, at least about 90%, or at least about 95% of the core fragments have less than four hydrogen bond acceptors, and at least about 50%, at least about 75%, at least about 90%, or at least about 95% of the core fragments have a calculated LogP of less than six, less than five, or less than four.

Libraries of the present invention may be virtual libraries, in that they are collections of computational or electronic representions of core fragments. The libraries may also be “wet” or physical libraries, in that they are collection of core fragments that are actually obtained through, for example, synthesis or purification, or they may be a combination of wet and virtual, with some of the core fragments having been obtained and others remaining virtual, or both. Libraries of the present invention may, for example, comprise at least about 10, at least about 50, at least about 100, at least about 500, at least about 750, at least about 1,000, or at least about 2,500 core fragments or compounds. Libraries of the present invention may, for example, comprise less than about 101, less than about 61, less than about 41, less than about 21, or less than about 11 core fragments or compounds. Libraries of the present invention may include subsets of larger libraries, comprising at least two members of the larger library. At least about 40%, at least about 50%, at least about 75%, or at least about about 90% of the core fragments of the libraries of the present invention, for example, comprise a handle comprising a substituent having anomalous dispersion properties. At least about 40%, at least about 50%, at least about 75%, at least about 90%, or at least about 95% of the core fragments of the libraries of the present invention have less than six, less than five, or, for example, less than four hydrogen bond acceptors. At least about 40%, at least about 50%, at least about 75%, at least about 90%, or at least about 95% of the core fragments of the libraries of the present invention have less than six, less than five, or, for example, less than four hydrogen bond donors. At least about 40%, at least about 50%, at least about 75%, at least about 90%, or at least about 95% of the core fragments or compounds of the libraries of the present invention have a calculated LogP value of less than six, less than five, or, for example, less than four. At least about 40%, at least about 50%, at least about 75%, at least about 90%, or at least about 95% of the core fragments or compounds of the libraries of the present invention have a molecular weight of less than about 350, for example, less than about 300, less than about 250, or less than about 200 daltons.

The present invention also provides linear compound libraries, or libraries comprising more than one linear library. For example, in one aspect is provided a linear compound library comprising a plurality of compounds, wherein, each compound comprises a central core and two or more handles, and wherein at least one of the handles comprises a substituent having anomalous dispersion properties. For example, at least about 50% of the compounds of the library have a molecular weight of less than about 300 daltons, or at least about 50% of the compounds have a core fragment comprising less than about five heteroatoms.

Linear libraries are also provided in aspects of the present invention. For example, in one aspect, a linear compound library is provided comprising a plurality of compounds, wherein, each compound comprises

a. the same central core;

b. n handles, wherein said handles are attached at the same positions on each compound; and

c. at least one derived substituent that differs from the derived substituent on another compound of said library;

wherein said derived substituent is derived from one handle and n+1 is an integer and less than or equal to the number of available bonds on the central core.

The derived substituents on the compounds may, for example, have been selected using computational methods. The derived substituents on said compounds may, for example, have been selected to have improved biological activity against a biological target molecule. The derived substituents may, for example, have been selected after a screening step; wherein said screening step comprises obtaining the structure of a core fragment in association with a biological target molecule. In one aspect, each of derived substituents is synthesized by modifying a first handle on said core fragment. In another aspect is provided a compound library comprising two or more linear compound libraries of the present invention.

Also included in the scope of the present invention are computer processor executable instructions on one or more computer readable storage devices wherein the instructions cause representation and/or manipulation, via a computer output device, of a core fragment library or compound library of the present invention. For example, the processor executable instructions are provided on one or more computer readable storage devices wherein the instructions cause representation and/or manipulation, via a computer output device, of a library of the present invention, such as, for example, a core fragment or compound library, the library may comprise a plurality of core fragments or compounds, wherein, each core fragment or compound comprises a central core and two or more handles, at least one of the handles comprises a substituent having anomalous dispersion properties, and wherein the handles can be readily modified using a one or two-step chemical synthesis process. The present invention also provides processor executable instructions on one or more computer readable storage devices wherein the instructions cause representation and/or manipulation, via a computer output device, of a combination of structures for analysis, where the combination comprises the structure of one or more members of a library of the present invention, and a biological target molecule. In one aspect of the invention the structure of the one or more member of the library can be represented or displayed as interacting with at least a portion of a substrate binding pocket structure of the biological target molecule. The processor executable instructions may optionally include one or more instructions directing the retrieval of data from a computer readable storage medium for the representation and/or manipulation of a structure or structures described herein.

Also provided in the present invention is a compound library comprising two or more sets of compounds, wherein each set of compounds comprises a central core and two or more handles, and wherein at least one of the handles comprises a substituent having anomalous dispersion properties.

In another aspect of the invention, combinations are provided. For example, provided in the present invention is a combination of structures for analysis, comprising a core fragment or compound library of the present invention, and a biological target molecule, wherein the structures comprise members of the library, the target molecule, and combinations thereof. Also provided in the present invention is a combination of structures for analysis, comprising a member of a core fragment or compound library of the present invention and a biological target molecule, wherein the structures comprise the library member, the biological target molecule, and combinations thereof. The combination may be virtual, for example, computational representations, or actual or wet, for example, physical entities. In one example, at least one member of the library binds to a portion of a ligand binding site of the target molecule. In some aspects of the combination, the concentration ratio of library members to target molecules is in a ratio of, for example about 50,000, about 25,000, about 10,000, about 1,000, about 100, or about 10 mol/mol. In some aspects of the combination, the concentration of library members is close to, at, or beyond the solubility point of the solution.

The present invention also provides a mixture for analysis by x-ray crystallography, comprising a plurality of core fragments or compounds selected from a library of the present invention and a biological target molecule. The biological target molecule, may, for example, be a protein, or a nucleic acid. The biological target molecule may, for example, be crystalline.

Methods of designing novel compounds or lead candidates are provided in the present invention. For example, in one aspect of the present invention, a method is provided of designing a lead candidate having activity against a biological target molecule, comprising obtaining a library of the present invention, determining the structures of one or more, and in some embodiments of the invention at least two, members of the library in association with the biological target molecule, and selecting information from the structures to design at least one lead candidate. The method may further comprise the step of determining the structure of the lead candidate in association with the biological target molecule. In one aspect, the method further comprises the step of designing at least one second library of compounds wherein each compound of the second library comprises a central core and two or more handles; and each compound of the second library differs from each other compound of the second library at at least one handle or derived substituent. In one aspect of the invention, the central core of the compounds of the second library and the central core of the lead candidate are the same. In one aspect, the method further comprises the steps of obtaining the second library; and determining the structures of one or more, and in some embodiments of the invention at least two, compounds of the second library in association with the biological target molecule. The biological target molecule may be, for example, a protein or, for example, a nucleic acid. The biological target molecule may, for example, be crystalline. The method may, for example, comprise preparing a plurality of mixtures of the biological target molecule with at least one of the core fragments. The method may, for example, comprise preparing a mixture of the biological target molecule with a plurality of the core fragments. The method may, for example, further comprise the step of assaying the biological activity of one or more, and in some embodiments of the invention at least two, core fragments against the biological target molecule. The assay may, for example, be a biochemical activity assay, or, for example, a biophysical assay, such as, for example, a binding assay, including, for example, but not limited to, an assay that comprises the use of mass spectroscopy. The biological activity assay may, for example, be conducted before, after, or simultaneously with obtaining the structure of the core fragment or compound in association with the biological target molecule. In one example, a subset of the core fragments or compounds assayed in the biological activity assay are selected for the structure determination step. In another example, a subset of the core fragments or compounds used in the structure determination step are assayed in the biological activity assay. In one aspect of the invention, the structure is determined using a method comprising X-ray crystallography. In one example, the method may further comprise the step of analyzing the binding of one or more, and in some embodiments of the invention at least two, core fragments to the biological target molecule using a computational method.

In one example, the method may further comprise the steps of selecting or otherwise using information about the structures to design at least one second library, wherein the second library is derived from at least one core fragment of the core fragment library; and comprises compounds having modifications on at least one of the handles on the core fragment. The second library may, for example, be a linear library, a plurality of linear libraries, or a combinatorial library. The method may, for example, further comprise the step of assaying the biological activity of one or more, and in some embodiments of the invention at least two, of the compounds against the biological target molecule.

The present invention also provides a method of designing a lead candidate having activity against a biological target molecule, comprising obtaining a mixture of the present invention, determining the structures of at least one compound of the mixture in association with the biological target molecule, and selecting information from the structure to design at least one lead candidate.

The present invention also comprises methods where the core fragment library may be screened against a first biological target molecule and eventually developed for activity against a second biological molecule. In some aspects of the invention, core fragments or compounds found to have activity toward one biological target molecule may be screened against other biological target molecules where they may, for example, have the same or even enhanced activity. The second biological target molecule may, for example, be a related protein, and may, for example, be from the same protein family, for example, a protease, phosphatase, nuclear hormone receptor, or kinase family. Thus, provided in the present invention is a method of designing a candidate compound having activity against a second biological target molecule, comprising obtaining a lead candidate of the present invention, determining the interaction of the lead candidate with a second biological target molecule; and designing at least one second library of compounds wherein each compound of the second library comprises a central core found in the lead candidate and modifications on at least one of the handles on the central core.

In other methods of the invention, the core fragment or compound libraries are used in binding or biological activity assays before crystallization, and those core fragments or compounds exhibiting a certain threshold of activity are selected for crystallization and structure determination. The binding or activity assay may also be performed at the same time as, or after, crystallization. Because of the ability to determine any complex structure, the threshold for determining whether a particular core fragment or compound is a hit may be set to be more inclusive than traditional high throughput screening assays, because obtaining a large number of false positives would not greatly negatively affect the process. For example, weak binders from a binding assay may be used in crystallization, and any false-positives easily weeded out. In other methods of the invention, the binding or biological activity assays may be performed after crystallization, and the information obtained, along with the structural data, used to determine the direction of the follow-up combinatorial library.

In one aspect of the present invention, derivative compounds are selected from each linear library, wherein each linear library comprises core fragments with modifications at one handle, resulting in a derived substituent, and for each linear library, the handle that is modified is a different handle, a new derivative compound is selected having the best-scoring handles in one compound. This selected derivative compound may be used as the basis of a new round of linear library design and screening, or may be the basis of a more traditional combinatorial library. The selected derivative compound may also be subjected to computational elaboration, in that it may serve as the basis for the individual design of an improved compound for screening. The cycle continues until a new derivative compound is obtained that may be considered to be a lead compound, having a desired IC₅₀, and other lead compound properties.

The present invention also provides methods for designing the core fragment and compound libraries of the present invention. Provided in the present invention is a method of designing a core fragment library for drug discovery, comprising screening or reviewing a list of synthetically accessible or commercially available core fragments, and selecting core fragments for the library wherein each of the core fragments comprises: two or more handles and less than 17 non-hydrogen atoms. The core fragments of the library may, for example, comprise, in their central core, at least one single or fused ring system. The core fragments of the library may, for example, comprise in their central core at least one hetero atom on at least one ring system.

Also provided in the present invention is a method of screening for a core fragment for use as a base core fragment for library design, comprising obtaining a library of the present invention, screening the library for members having binding activity against a biological target molecule; and selecting a core fragment of member(s) with binding activity to use as a base core fragment for library design.

Also provided in the present invention are lead candidates and candidate compounds obtained by the methods of the present invention, libraries obtained by the methods of the present invention, and libraries comprising compounds with core fragments selected by the methods of the present invention.

The present invention also provides a method of designing a lead candidate having biophysical or biochemical activity against a biological target molecule, comprising obtaining the structure of the biological target molecule bound to a core fragment or compound, wherein the core fragment or compound comprises a substituent having anomalous dispersion properties, synthesizing a lead candidate molecule comprising the step of replacing a handle or derived substituent on the compound with a substituent comprising a functionalized carbon, nitrogen, oxygen, sulfur, or phosphorus atom, and assaying the lead candidate molecule for biophysical or biochemical activity against the biological target molecule. In some aspects, the anomalous dispersing atom, such as Br, may be found to assist in binding to the biological target molecule. In this case, the atom may also be present on the second substituent, and in some aspects, the handle comprising the substituent having anomalous dispersion properties remains, while another handle is modified or replaced.

The present invention also provides a method of designing a lead candidate having biophysical or biochemical activity against a biological target molecule, comprising combining a biological target molecule with a mixture comprising one or more, and in some embodiments of the invention at least two, core fragments or compounds, wherein at least one of the core fragments or compounds comprises a substituent having anomalous dispersion properties, identifying a core fragment or compound bound to the biological target molecule using the anomalous dispersion properties of the substituent, synthesizing a lead candidate molecule comprising the step of replacing the anomalous dispersion substituent with a substituent comprising a functionalized carbon or nitrogen atom, and assaying the lead candidate molecule for biophysical or biochemical activity against the biological target molecule.

The present invention takes advantage of the ability to rapidly obtain the structures of target proteins complexed with test compounds, and the ability of using rapid and accessible synthetic chemistry.

In one embodiment of the invention, an initial library of core fragments is designed, by selecting core fragments from available compound fragments, including those synthesized by or on behalf of the researcher, and those available from commercial libraries. The initial core fragment library is comprised of core fragments wherein, for example, at least about 25%, for example, at least about 40%, for example, at least about 50%, for example, at least about 60%, for example, at least about 70%, for example, at least about 80%, for example, at least about 90% of the core fragments have a molecular weight of less than about 250D. The initial core fragment library is comprised of core fragments wherein, for example, at least about 25%, for example, at least about 40%, for example, at least about 50%, for example, at least about 60%, for example, at least about 70%, for example, at least about 80%, for example, at least about 90% of the core fragments have less than about five heteroatoms. The initial core fragment library is comprised of core fragments wherein, for example, at least about 25%, for example, at least about 40%, for example, at least about 50%, for example, at least about 60%, for example, at least about 70%, for example, at least about 80%, for example, at least about 90% of the core fragments comprise a substituent that is capable of anomalous scattering, for example, but not limited, to Br. In one aspect, the initial core fragment library is comprised of core fragments wherein, for example, at least about 25%, for example, at least about 40%, for example, at least about 50%, for example, at least about 60%, for example, at least about 70%, for example, at least about 80%, for example, at least about 90% of the core fragments contain Br. The initial core fragment library is comprised of core fragments wherein, for example, at least about 25%, for example, at least about 40%, for example, at least about 50%, for example, at least about 60%, for example, at least about 70%, for example, at least about 80%, for example, at least about 90% of the core fragments comprise handles. Core fragments in the library may, independently, comprise, for example, about two, about three, about four, or about five or more handles.

Definitions

A “core fragment” or “core moiety” is a molecule, or part thereof, selected or designed to be part of a synthetic precursor to lead candidate or drug candidate. A core fragment comprises one, two, or three or more chemical substituents, also called “handles”. A core fragment preferably exhibits properties of desirable lead compounds, including, for example, a low molecular complexity (low number of hydrogen bond donors and acceptors, low number of rotatable bonds, and low molecular weight), and low hydrophobicity. Because the core fragment is small, one of ordinary skill in the art may further develop or elaborate the core fragment into a lead or drug candidate by modifying the core fragment, either at the handles or at the core structure, to have desirable drug characteristics, including, for example, by meeting the Lipinski rule of five. Preferred core fragment properties include lead-like properties and are known to those of ordinary skill in the art and are described in Teague, S. J., et al., Agnew. Chem. Int. Ed. 38:3743-3748, 1999; Oprea, T. I., et al., J. Chem. Inf. Comput. Sci. 41:1308-1315, 2001; and Hann, M. M. et al., J. Chem. Inf. Comput. Sci. 41:856-864, 2001. Desirable core fragments include, but are not limited to, for example, molecules having many or all of the following general properties: Mᵣ<about 350, <about 300, or <about 250, a clogP<about 3, less than about 5 rings, and an LogP<about 5 or <about 4. Other general properties may include less than about 11 nonterminal single bonds, less than about 6 hydrogen bond donors, and less than about 9 hydrogen bond acceptors. Thus, core fragments are designed so that more complexity and weight may be added during development and building out of the compound into a lead candidate, while maintaining the general properties.

Core fragments may comprise central cores comprising cyclic or non-cyclic structures. A core fragment may be, for example, and not limited to, and for purposes of illustration only, a molecule such as one of the following, with handles circled:

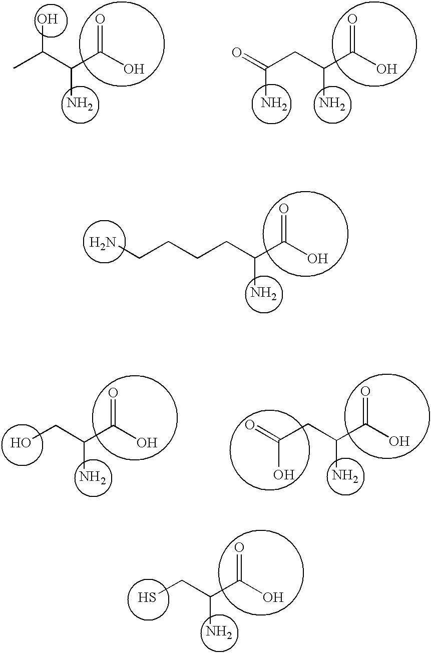

Other examples of non-cyclic central cores, include, but are not limited to, hypusine, putrescine, gamma-aminobutyric acid, and 2-hydroxyputresine.

Alternatively, the non-handle portion of a core fragment may comprise 1) a cyclic structure, including any of the cyclic structures described herein, with 2) one or more of the handles disclosed herein. Therefore, cyclic structures comprising, but not limited to, anyone or more of the handles illustrated above are within the scope of “core fragment.”

A “central core” or “core scaffold” is a molecule that generally does not include handles, as described herein, but may include internal handles, such as atoms that are part of one of the central rings. A core fragment comprises a central core and at least one handle. Non-limiting examples of a central core include any cyclic or non-cyclic structure, such as, but not limited to, those disclosed herein. In some embodiments of the invention, a central core is the portion of a core fragment lacking one or more handles. Compounds of the invention include those comprising a central core and one or more handles. A central core preferably exhibits properties of desirable lead compounds, including, for example, a low molecular complexity (low number of hydrogen bond donors and acceptors, low number of rotatable bonds, and low molecular weight), and low hydrophobicity. Because a central core is small, one of ordinary skill in the art may further develop or elaborate the core into a lead or drug candidate by modifying the core to have desirable drug characteristics, including, for example, by meeting the Lipinski rule of five. Preferred core properties include lead-like properties and are known to those of ordinary skill in the art and are described in Teague, S. J., et al., Agnew. Chem. Int. Ed. 38:3743-3748, 1999; Oprea, T. I., et al., J. Chem. Inf. Comput. Sci. 41:1308-1315, 2001; and Hann, M. M. et al., J. Chem. Inf. Comput. Sci. 41:856-864, 2001. Desirable central cores include, but are not limited to, for example, molecules having many or all of the following general properties: Mᵣ<about 350, <about 300, or <about 250, a clogP<about 3, less than about 5 rings, and an LogP<about 5 or <about 4. Other general properties may include less than about 11 nonterminal single bonds, less than about 6 hydrogen bond donors, and less than about 9 hydrogen bond acceptors. Thus, central cores are designed so that more complexity and weight may be added during development and building out of the molecule into a lead candidate, while maintaining the general properties.

A “handle” is a functional chemical group, or substituent, covalently attached to a site on a core fragment or central core at which various reactive groups may be substituted or added. Handles are used for bond-forming reactions. For example, a carbon atom that is part of the central core may be bound to a methyl group handle. This site may also be within the central core; for example, a hydrogen atom on a carbon atom within a ring may be a handle. Handles can preferably be modified or replaced by other handles or derived substituents using one step or two step chemical processes. Protection and de-protection steps may also be required. In an aspect of the invention, this modification may be done independently at each handle, without the need to add protecting groups at the other handles. Handles may comprise substituents capable of anomalous scattering.

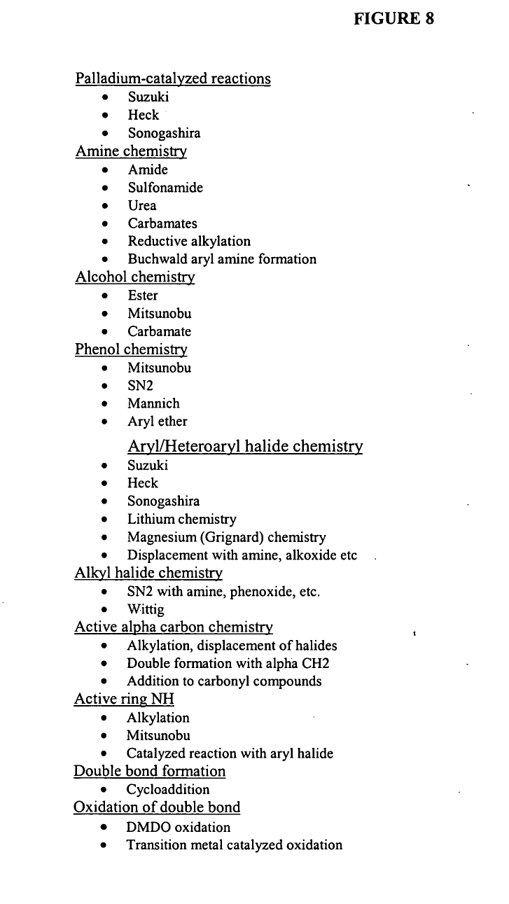

Reactions useful for one-or two-step synthesis include for example, but are not limited to, examples presented in FIG. 8, and include, for example, Suzuki coupling, Heck coupling, Sonogashira coupling, Wittig reaction, alkyl lithium-mediated condensations, halogenation, SN2 displacements (for example, N, O, S), ester formation, and amide formation, as well as other reactions that may be used to generate handles such as those presented herein. Other reactions are provided, for example, in FIG. 8. Reactions may also. be selected based on the ease of purification required.

Handles that may be used in some aspects of the present invention include, but are not limited to H, benzyl halide, benzyl alcohol, allyl halide, allyl alcohol, carboxylic acid, aryl amine, heteroaryl amine, benzyl amine, aryl alkyl amine, alkyl amino, phenol, aryl halide, heteroaryl halide, heteroaryl chloride, aryl aldehyde, heteroaryl aldehyde, aryl alkyl aldehyde, alkyl aldehyde, aryl, heteroaryl, alkyl, aryl alkyl, ketone, arylthiol, heteroaryl thiol, urea, imide, aryl boronic acid, ester, carbamate, tert-butyl carbamate, nitro, aryl methyl, heteroaryl methyl, vinyl methyl, 2-or 2,2-substituted vinyls, 2-substituted alkynes, acyl halide, aryl halide, alkyl halide, cycloalkyl halide, sulfonyl halide, carboxylic anhydride, epoxide, and sulfonic acid. In some embodiments, the handles may include, but are not limited to benzyl bromide, benzyl alcohol, allyl bromide, allyl alcohol, carboxylic acid, aryl amine, heteroaryl amine, benzyl amine, aryl alkyl amine, phenol, aryl bromide, heteroaryl bromide, heteroaryl chloride, aryl aldehyde, heteroaryl aldehyde, aryl alkyl aldehyde, ketone, arylthiol, heteroaryl thiol, urea, imide, and aryl boronic acid. Halide may include, for example, iodide, bromide, fluoride, and chloride. Halide may include halides capable of anomalous scattering, such as, for example, bromide or iodide.

Handles embodied in the present invention include, but are not limited to those listed in Table 1. By convention, these handles may be considered as either “direct” handles or “latent” handles, with some having the capacity to function as either, which are indicated as “both” in Table 1. A direct handle is a functional group or moiety that can react directly with another functional group or moiety without prior modification or that can be rendered reactive by the addition of reagents and/or catalysts typically, but not necessarily, in a single-pot reaction. Examples of a direct handle include, but are not limited to: the Br in a benzyl bromide, carboxylic acid, amine, phenol, the Br in an aryl bromide, aldehyde, thiol, boronic acid or ester, and the like. A latent handle is a functional group or moiety that requires prior modification, either in a separate step after which it may or may not be isolated, or generated in situ to afford a more reactive species (i.e., obtaining a direct handle). A latent handle may also comprise a moiety that by virtue of its proximity or connectivity to a functional group or other moiety is rendered reactive. Examples of a latent handle include, but are not limited to: nitro (which can be reduced to an amine), aryl methyl (which can be converted to aryl bromomethyl or to aryl carboxylic acid), olefin (which can undergo oxidative cleavage to afford an epoxide, an aldehyde or carboxylic acid), and the like. The adoption of the above convention serves to illustrate the scope of chemical moieties regarded as handles within the present invention and it is clearly not limited to those shown in Table 1. Additional handles are within the scope of this invention and are evident to those trained in the art and having access to the chemical literature.

TABLE 1

| entry | Type | Handle | Comment |
|---|---|---|---|
| 1 | direct | —NHR | R = H, alkyl, substituted alkyl, aryl, heteroaryl, hetrocyclyl |
| 2 | direct | —YH | Y = O, S |
| 3 | both | —X | X = Br, Cl, SR, SOR, SO2R, OH, OC(O)R, OC(O)OR; R = alkyl, substituted |
| | | | alkyl, aryl, heteroaryl, hetrocyclyl |
| 4 | direct | —(CH₂)—X | X = Br, Cl, I, SR, SOR, SO2R, OH, OC(O)R, OC(O)OR, C(O)R, C(O)OR; R = |
| | | | alkyl, substituted alkyl, aryl, heteroaryl, hetrocyclyl |
| 5 | both | —C(X)R | X = O, NOR; R = H, alkyl, substituted alkyl, aryl, heteroaryl, hetrocyclyl |
| 6 | both | —C(X)YR | X = O, NR; Y = O, NH, NMe; R = H, alkyl, substituted alkyl, aryl, heteroaryl, |
| | | | hetrocyclyl, alkoxy (when Y = NMe) |
| 7 | both | —CN | |
| 8 | both | —NHC(X)R | R = H, alkyl, substituted alkyl, aryl, heteroaryl, hetrocyclyl, OR′, NR′R″; R,R″ = |
| | | | alkyl, substituted alkyl, aryl, heteroaryl; X = O, S |
| 9 | direct | 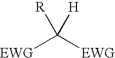 | EWG = CN, COR, CONR2, COOR, CHO, NO2, aryl, heteroaryl; R = H, alkyl, aryl, heteroaryl; point of attachment at R or EWG |
| 10 | direct | —SO₂NHR | R = H, alkyl, substituted alkyl, aryl, heteroaryl, hetrocyclyl |
| 11 | direct | 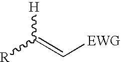 | EWG = CN, COR, COOR, CHO, NO2; R = alkyl, aryl, heteroaryl; point of attachment at R or EWG |
| 12 | direct | 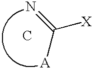 | X = F, Cl, SR, SO2R; A = S, O, NR; R = alkyl, aryl; ring C contained in core |
| 13 | direct | 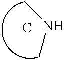 | C = ring contained in core |
| 14 | direct | 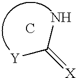 | Y = bond, O, S, NR, C(X); X = O, S; R = alkyl, aryl, heteroaryl; C = ring contained in core |
| 15 | direct | —B(OH)₂ | |
| 16 | both | —NO₂ | |
| 17 | latent | Ar—CH₃ | Ar = aryl, heteroaryl, heterocyclyl ring contained in core |
| | latent | Ar—H | Ar = aryl, heteroaryl, heterocyclyl ring contained in core |
| 18 | both | —C(O)CH₃ | |
| 19 | both | 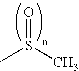 | n = 0, 1, 2 |
| 20 | latent | 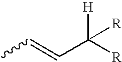 | Allylic hydrogen; R = H, alkyl |
| 21 | both | 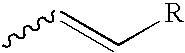 | Olefin; R = H, alkyl, aryl, heteroaryl, alkoxy, alkylamino |
| 22 | both | —≡—R | Alkyne; R = H, alkyl, aryl, heteroaryl |
| 23 | latent | 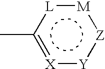 | X, Y, Z, M, L selected from C, N, O, S, bond; 4-6 membered rings which undergo thermolysis or photolysis to afford reactive intermediates |

A “derived substituent” is a substituent on a compound that is derived from a handle. The derived substituent may be, for example, a modified handle, where some of the atoms of the original handle remain. Or, the derived substituent may be completely modified by substituting or replacing a handle with a different substituent. Or, the derived substituent may be modified by adding substituents to a handle. Derived substituents are, for example, derived from handles on core fragments. A derived substituent may be capable of modification, substitution or replacement using a one or two step synthetic process such as those, for example, presented herein, but not including potential protection or deprotection steps. Or, a derived substituent may not be capable of modification, substitution or replacement using a one or two step synthetic process. Derived substituents capable of further modification, substitution or replacement may of course be subjected to such reactions as deemed desirable by the skilled person practicing the invention.

“Single ring” refers to a cycloalkyl, heterocycloalkyl, aryl, or heteroaryl ring having about three to about eight, or about four to about six ring atoms. A single ring is not fused by being directly bonded at more than one ring atom to another closed ring.

“Fused ring” refers to fused aryl or cyclyl ring. For example, about six or less, about five or less, about four or less, about three or less, or about two rings may be fused. Each ring may be independently selected from the group consisting of aryl, heteroaryl, cycloalkyl, heterocycloalkyl, cycloalkenyl, and heterocycloalkenyl rings, each of which ring may independently be substituted or unsubstituted, having about four to about ten, about four to about thirteen, or about four to about fourteen ring atoms.

The number of rings in a central core or core fragment refers to the number of single or fused ring systems. Thus, for example a fused ring may be considered to be one ring. As non-limiting examples, a phenyl ring, naphthalene, and norbomane, for purposes of the present invention, are all considered to be one ring, whereas biphenyl, which is not fused, is considered to be two rings.

A “heteroatom” refers to N, O, S, or P. In some embodiments, heteroatom refers to N, O, or S, where indicated. Heteroatoms shall include any oxidized form of nitrogen, sulfur, and phosphorus and the quaternized form of any basic nitrogen.

A “library” is a collection of core fragments or compounds. The library may be virtual, in that it is an in silico or electronic collection of structures used for computational analysis as described herein. The library may be physical, in that the set of core fragments or compounds are synthesized, isolated, or purified.

A “lead candidate” is a compound that binds to a biological target molecule and is designed to modulate the activity of a target protein. A lead candidate may be used to develop a drug candidate, or a drug to be used to treat a disorder or disease in an animal, including, for example, by interacting with a protein of said animal, or with a bacterial, viral, fungal, or other organism that may be implicated in said animal disorder or disease, and that is selected for further testing either in cells, in animal models, or in the target organism. A lead candidate may also be used to develop compositions to modulate plant diseases or disorders, including, for example, by modulating plant protein activity, or by interacting with a bacterial, viral, fungal, or other organism implicated in said disease or disorder.

A “drug candidate” is a lead candidate that has biological activity against a biological target molecule and has ADMET (absorption, distribution, metabolism, excretion and toxicity) properties appropriate for it to be evaluated in an animal, including a human, clinical studies in a designated therapeutic application.

A “compound library” is a group comprising more than one compound, used for drug discovery. The compounds in the library may be compound fragments, designed to be linked to other compound fragments, or the compounds may be larger compounds, designed to be used without linkage to other compounds.

A “plurality” is more than one of whatever noun “plurality” modifies in the sentence.

The term “obtain” refers to any method of obtaining, for example, a core fragment, a compound, biological target molecule, or a library. The method used to obtain such core fragment, compound, biological target molecule, or library, may comprise synthesis, purchase, or any means the core fragment, compound, biological target molecule, or library can be obtained.

By “activity against” is meant that a compound may have binding activity by binding to a biological target molecule, or it may have an effect on the enzymatic or other biological activity of a target, when present in a target activity assay. Biological activity and biochemical activity refer to any in vivo or in vitro activity of a target biological molecule. Non-limiting examples include the activity of a target molecule in an in vitro, cellular, or organism level assay. As a non-limiting example with an enzymatic protein as the target molecule, the activity includes at least the binding of the target molecule to one or more substrates, the release of a product or reactant by the target molecule, or the overall catalytic activity of the target molecule. These activities may be accessed directly or indirectly in an in vitro or cell based assay, or alternatively in a phenotypic assay based on the effect of the activity on an organism. As a further non-limiting example wherein the target molecule is a kinase, the activity includes at least the binding of the kinase to its target polypeptide and/or other substrate (such as ATP as a non-limiting example) as well as the actual activity of phosphorylating a target polypeptide.

Obtaining a crystal of a biological target molecule in association with or in interaction with a test core fragment or compound includes any method of obtaining a compound in a crystal, in association or interaction with a target protein. This method includes soaking a crystal in a solution of one or more potential compounds, or ligands, or incubating a target protein in the presence of one or more potential compounds, or ligands.

A core fragment, handle, halide, substituent, or molecule is “capable of anomalous dispersion” or anomalous scattering, when it contains an atom that exhibits absorption of incident x-rays of a wavelength that is experimentally accessible either from a conventional x-ray source or a synchrotron. Examples include, but are not limited to bromine, including bromo-derivatives, iodine, selenium, and sulfur, for example in the form of SH or SR, where R is a functional group.

By “or” is meant one, or another member of a group, or more than one member. For example, A, B, or C, may indicate any of the following: A alone; B alone; C alone; A and B; B and C; A and C; A, B, and C.

“Association” refers to the status of two or more molecules that are in close proximity to each other. The two molecules may be associated non-covalently, for example, by hydrogen-bonding, van der Waals, electrostatic or hydrophobic interactions, or covalently.

“Active Site” refers to a site in a target protein that associates with a substrate for target protein activity. This site may include, for example, residues involved in catalysis, as well as residues involved in binding a substrate. Inhibitors may bind to the residues of the active site.

“Binding site” refers to a region in a target protein, which, for example, associates with a ligand such as a natural substrate, non-natural substrate, inhibitor, substrate analog, agonist or antagonist, protein, co-factor or small molecule, as well as, optionally, in addition, various ions or water, and/or has an internal cavity sufficient to bind a small molecule and may be used as a target for binding drugs. The term includes the active site but is not limited thereby.

“Crystal” refers to a composition comprising a biological target molecule, including, for example, macromolecular drug receptor targets, including protein, including, for example, but not limited to, polypeptides, and nucleic acid targets, for example, but not limited to, DNA, RNA, and ribosomal subunits, and carbohydrate targets, for example, but not limited to, glycoproteins, crystalline form. The term “crystal” includes native crystals, and heavy-atom derivative crystals, as defined herein. The discussion below often uses a target protein as a exemplary, and non-limiting example. The discussion applies in an analogous manner to all possible target molecules.

By “modification” (or modify) at a handle is meant to include synthetic modifications to the handle itself, or an exchange of the handle with another handle or derived substituent. A modified handle may itself be a handle, that can be modified or replaced using a one-step or two-step chemical process. A modified handle may be a substituent that is not as easily modified or replaced.

“Alkyl” and “alkoxy” used alone or as part of a larger moiety refers to both straight and branched chains containing about one to about eight carbon atoms. “Lower alkyl” and “lower alkoxy” refer to alkyl or alkoxy groups containing about one to about four carbon atoms.

“Cyclyl”, “cycloalkyl”, or “cycloalkenyl” refer to cyclic alkyl or alkenyl groups containing from about three to about eight carbon atoms. “Lower cyclyl,” “lower cycloalkyl.” or “lower cycloalkenyl” refer to cyclic groups containing from about three to about six carbon atoms.

“Alkenyl” and “alkynyl” used alone or as part of a larger moiety shall include both straight and branched chains containing about two to about eight carbon atoms, with one or more unsaturated bonds between carbons. “Lower alkenyl” and “lower alkynyl” include alkenyl and alkynyl groups containing from about two to about five carbon atoms.

“Halogen” means F, Cl, Br, or I.

“Aryl”, used alone or as part of a larger moiety as in “aralkyl”, refers to aromatic rings having six ring carbon atoms.

“Fused aryl,” refers to fused about two to about three aromatic rings having about six to about ten, about six to about thirteen, or about six to about fourteen ring carbon atoms.

“Fused heteroaryl” refers to fused about two to about three heteroaryl rings wherein at least one of the rings is a heteroaryl, having about five to about ten, about five to about thirteen, or about five to about fourteen ring atoms.

“Fused cycloalkyl” refers to fused about two to about three cycloalkyl rings having about four to about ten, about four to about thirteen, or about four to about fourteen ring carbon atoms.

“Fused heterocycloalkyl” refers to fused about two to about three heterocycloalkyl rings, wherein at least one of the rings is a heterocycloalkyl, having about four to about ten, about four to about thirteen, or about four to about fourteen ring atoms.

“Heterocycloalkyl” refers to cycloalkyls comprising one or more heteroatoms in place of a ring carbon atom.

“Lower heterocycloalkyl” refers to cycloalkyl groups containing about three to six ring members.

“Heterocycloalkenyl” refers to cycloalkenyls comprising one or more heteroatoms in place of a ring carbon atom. “Lower heterocycloalkenyl” refers to cycloalkyl groups containing about three to about six ring members. The term “heterocycloalkenyl” does not refer to heteroaryls.

“Heteroaryl” refers to aromatic rings containing about three, about five, about six, about seven, or about eight ring atoms, comprising carbon and one or more heteroatoms.

“Lower heteroaryl” refers to heteroaryls containing about three, about five, or about six ring members.

“Linker group” means an organic moiety that connects two parts of a compound. Linkers are typically comprised of an atom such as oxygen or sulfur, a unit such as —NH— or —CH₂—, or a chain of atoms, such as an alkylidene chain. The molecular mass of a linker is typically in the range of about 14 to about 200. Examples of linkers are known to those of ordinary skill in the art and include, but are not limited to, a saturated or unsaturated C₁₋₆ alkylidene chain which is optionally substituted, and wherein up to two saturated carbons of the chain are optionally replaced by —C(═O)—, —CONH—, CONHNH—, —CO₂—, —NHCO₂—, —O—, —NHCONH—, —O(C═O)—, —O(C═O)NH—, —NHNH—, —NHCO—, —S—, —SO—, —SO₂—, —NH—, —SO₂NH—, or NHSO₂—.

The term “N-protected amino” refers to protecting groups intended to protect an amino group against undesirable reactions during synthetic procedures. Commonly used N-protecting groups are disclosed in Greene, “Protective Groups In Organic Synthesis,” (John Wiley & Sons, New York (1981)). Preferred N-protecting groups are formyl, acetyl, benzoyl, pivaloyl, t-butylacetyl, phenylsulfonyl, benzyl, t-butyloxycarbonyl (Boc), and benzyloxycarbonyl (Cbz).

The term “O-protected carboxy” refers to a carboxylic acid protecting ester or amide group typically employed to block or protect the carboxylic acid functionality while the reactions involving other functional sites of the compound are performed. Carboxy protecting groups are disclosed in Greene, “Protective Groups in Organic Synthesis” (1981). Additionally, a carboxy protecting group can be used as a prodrug whereby the carboxy protecting group can be readily cleaved in vivo, for example by enzymatic hydrolysis, to release the biologically active parent. Such carboxy protecting groups are well known to those skilled in the art, having been extensively used in the protection of carboxyl groups in the penicillin and cephalosporin fields as described in U.S. Pat. Nos. 3,840,556 and 3,719,667.

An LogP value may be, for example, a calculated Log P value, for example, one determined by a computer program for predicting Log P, the log of the octanol-water partition coefficient commonly used as an empirical descriptor for predicting bioavailability (e.g. Lipinski's Rule of 5; Lipinski, C. A.; Lombardo, F.; Dominy, B. W.; Feeney, P. J. (1997) Experimental and computational approaches to estimate solubility and permeability in drug discovery and development settings. Adv. Drug Delivery Rev. 23, 3-25). The calculated logP value may, for example, be the SlogP value. SlogP is implemented in the MOE software suite from Chemical Computing Group, www.chemcomp.com. SlogP is based on an atomic contribution model (Wildman, S. A., Crippen, G. M.; Prediction of Physicochemical Parameters by Atomic Contributions; J. Chem. Inf. Comput. Sci., 39(5), 868-873 (1999)).

### EXAMPLE 1

### Selection and Design of Core Fragment Libraries

Selection of Core fragments

Computational methods may be used to select core fragments having handles that may be used in the present invention. Core fragments may be selected by searching, for example, available commercial chemical libraries. Core fragments are selected to have a desired molecular weight, for example a molecular weight of approximately 150-250 Daltons. Handles are selected to be amenable to synthesis of numerous combinations of modifications or replacement with other handles.

In one example of the methods of the present invention, the following procedure is followed. A library of compounds, for example, a commercially available library is searched to select compounds that are appropriate for further elaboration. Criteria for selection include, but are not limited to: 2-3 handles, and lead-like properties, such as, for example, a molecular weight below 250 D, and less than five heteroatoms. Some of the compounds, may, for example, comprise Br.

Using a structural model of the target protein, and/or the target protein binding site, a limited group of small molecules are selected as the starting points, the core fragments for selected library design. Each of the compound fragments may be designed, using computational methods known to those of ordinary skill in the art, to dock into a part of a target protein binding site. The initial target protein structural model may be an apostructure, or may be a complex. In another method of the present invention, the core fragment library is selected without docking or screening against a particular target molecule. In some aspects, the core fragment library is selected without being directed toward a particular target molecule.

### EXAMPLE 2

### Screening of Core Fragment Libraries

Once an initial core fragment library is selected, it may be used in screening for a core fragment that binds to, or has activity against, a particular biological target molecule, such as, for example, a protein or a nucleic acid. The core fragments may be first screened using a biochemical or biophysical assay, and then some or all of the core fragments may be used in structure determination methods. Or, the core fragments may be first screened using structure determination methods, with some or all of the core fragments then screened in biochemical or biophysical assays. Or, the two procedures may be simultaneous. Biophysical assays may be any assays that can measure the association between a core fragment or compound and a biological target molecule, while biological assays may be used, for example, to measure the IC₅₀ as being in a millimolar, micromolar, nanomolar, or picomolar range. Biophysical assays may include, but are not limited to, mass spectroscopic methods, and binding affinity methods known to those of ordinary skill in the art. The core fragment library is used to screen for core fragments that bind to the target protein, using, for example, X-ray crystallography. In one aspect of the invention, the core fragments are subjected to crystallization experiments in the presence of the target protein. The core fragments may, for example, be screened as mixtures, with at least two core fragments present in the crystallization mixture. The crystallization screening may comprise, for example, soaking of a crystal comprising the target in a solution comprising the test core fragment or core fragments. The crystallization screening may comprise, for example, the mixing of the target protein with the core fragment or core fragments, followed by crystallization. A biochemical assay or other activity assay may also be conducted either before, at the same time as, or after the binding assay. Each core fragment having the desired binding ability, and, if performed, the desired biochemical or other activity assay results, is selected alone, or in combination with other one or more other core fragments, as the basis for constructing a linear library.

Each of the experimentally, for example crystallographically, biophysically or biochemically, selected core fragments is expanded into a small virtual compound library of molecules, or derivative compounds, that can be readily synthesized from the core fragment. In one method of the present invention, in silico (or computer implemented) libraries are designed comprising core fragments comprising modified handles. In one method, the libraries are designed to be linear libraries, with each library comprising derivative compounds wherein, for example, at least about 25%, at least about 40%, at least about 50%, at least about 60%, at least about 70%, at least about 80%, at least about 90% of the derivative compounds differ from another derivative compound in the library at only one of the handles. These libraries are computationally screened to determine if they may bind at the desired binding site of the target. Those with desired binding characteristics are obtained, including, for example, by synthesis, and tested using, for example, crystallographic experiments and biophysical or biochemical assays Selected core fragments are then screened by X-ray crystallography and biochemically, to determine which of the selected core fragments should be used as a basis for a linear library. The screening may, for example, be conducted in any order. In one method, the core fragments are first screened in a biochemical assay for activity against a target. Those of ordinary skill in the art may select the appropriate biochemical assay for the intended target. Then, the core fragments that meet a certain threshold of activity, as determined for each target, are subjected to X-ray crystallographic analysis after crystallization with the target. Core fragments that bind to the intended active site of the target are then selected for linear library synthesis. In another method, the X-ray crystallographic analysis is conducted first, and those core fragments that bind to the target's active site are then subjected to biochemical assays. In the third method, the two screening procedures are conducted simultaneously, or at different times, with most, or all, of the core fragments being subjected to both procedures, and those core fragments having the desired activity and binding characteristics are selected as core fragments used for linear library synthesis.

Selecting core fragments having handles capable of anomalous scattering has several advantages, including, but not limited to the following. First, having a signal from anomalous dispersion can dramatically increase the sensitivity of the crystal structure determination technique, so that weak and/or significantly disordered ligands can be detected in cases where the ordinary difference electron density for the ligand would be ambiguous. Second, it makes it easier to automate the process of detecting bound ligands. Both of these advantages help make crystallographic screening a more viable alternative to conventional assay techniques.

The core fragments or compounds may optionally be screened, for activity as, for example, described herein, either before or after obtaining structural data. In the initial screening, each of the libraries is screened in one or more assays, including binding and/or biological assays. The core fragments or compounds exhibiting the most activity are then selected for further analysis. Core fragments or compounds may be selected with IC₅₀ values in the high micromolar range or lower, because their potential as leads will be confirmed through further analysis. Another method of initial screening to determine if the core fragment or compound is bound is by soaking the protein crystals in the presence of core fragments or compounds with anomalous scattering properties.

### EXAMPLE 3

### Design and Screening of Secondary (Linear or Combinatorial) Drug-Like Compound Libraries

Once a core fragment library is screened, and at least one core fragment is identified as having particular assay results and/or particular interactions with the biological target molecule, the core fragment may be elaborated to develop a secondary (linear or combinatorial) library for further screening and optimization. If no core fragments are identified as having desirable characteristics, the screening step may be repeated either by screening the same, or a different core fragment library.

The compounds of a secondary library selected for having the most activity are then analyzed using structural analysis of target/compound complexes. The selected compounds are subjected to crystallization with the target protein, either through soaking or through incubation with the target protein during initial crystallization. The crystals may be obtained by soaking the target protein crystals in the presence of one, or a mixture of compounds. Similarly, the crystals may be obtained by incubating the target protein in the presence of one, or a mixture of compounds. X-ray diffraction data are then collected from the crystals, and the data are analyzed to solve the structures. The most important functional groups are then selected for further analysis.

Once compounds are selected from a linear or combinatorial library, having desired activity and binding characteristics, they may be used for further elaboration. As a non-limiting example, a selected compound may serve as the basis for a tertiary linear library having changes at a different, handle, or at the same handle, or for a more traditional combinatorial library as the tertiary library. Or, compounds selected from more than one linear library may be combined and used to form the basis for a tertiary linear library, or combinatorial library. For example, a compound selected having handles A′BC, where A′ is a derived substituent, may be combined with a compound having handles ABC′, where C′ is a derived substituent, used as a basis for an additional linear library at either A′, B, or C′, or a combinatorial library having changes at any or all three handles. As each round of synthesis and screening are conducted, compounds are selected having more drug-like characteristics, with lower IC₅₀s, and other more desirable characteristics.

The secondary library may be a linear library or a combinatorial library. In one aspect of the invention, a linear library is developed, including a series of related compounds that are modified at only one of the handles. A compound screening library may include one, or more than one, linear library. In each linear library, the compounds have the same central core, and, if there is more than one handle, all except one of the handles comprise the same substituents. As a non-limiting example, for each core fragment, the synthetic handle(s) are used to create a small selected library. At each handle, multiple compounds are made using the handle as a convenient method of synthesis. For each library, most of the compounds differ from each of the other compounds by changes at one handle. For example, where a druglike compound is designed to include three handles, each of the handles may, for example, have ten different groups synthetically attached. Linear libraries comprising compounds with the same central core with modifications at, for example, one handle at a time, may be obtained. The linear libraries are then screened for binding to the target, using, for example, X-ray crystallography, and, may also be screened biochemically or in another activity assay. Compounds having desired binding and, for example, biochemical, activity, are then selected for further drug design, such as, but not limited to, the use of all or a part of a compound as the central core for development of additional compounds. Linear libraries may be constructed and tested all at once, or over a period of time. In this type of library, the members of the library would include, for example, those of Table 2, depicting a core fragment having three handles, A, B, and C. Each handle may have, for example, ten different groups attached. In this type of linear library, only one handle is modified at a time, as shown in Table 2. In this example, the modifications are at handle C. In another example, the modifications remain at handle C but handles at A and B remain constant with a different substituent, such as, but not limited to, one of the substituents shown below for handle C. In other analogous library example, the handle at C and B would remain constant, and the modifications would be at handle A, or the handles at A and C would be constant and the modifications would be at handle B.

TABLE 2

| Handle A | Handle B | Handle C | Compound # |
|---|---|---|---|
| 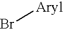 | 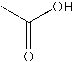 | 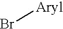 | 1 |
| 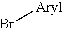 | 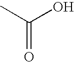 | 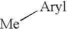 | 2 |
| 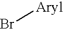 | 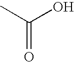 | 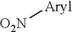 | 3 |
| 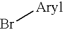 | 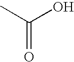 | 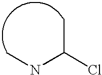 | 4 |
| 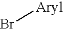 | 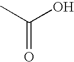 | 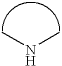 | 5 |
| 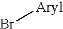 | 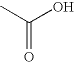 | 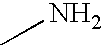 | 6 |
| 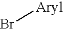 | 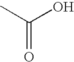 | 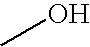 | 7 |
| 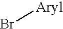 | 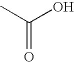 | 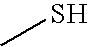 | 8 |
| 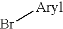 | 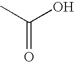 | 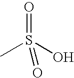 | 9 |
| 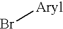 | 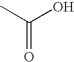 | 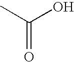 | 10 |

Using this non-limiting example of the method, multiple compound libraries, each having 10 different handles at one handle site is used in the initial screens. Once the linear libraries are synthesized, using a relatively low number of total compounds in each library, for example, less than 11, less than 21, less than 31, less than 51, and less than 101 compounds, the compounds are used for crystallization with the target, for example, either by mixing the target protein with the compound or a mixture of compounds, and then crystallizing the complex, or by soaking the target protein crystal in a solution comprising the compound or a mixture of compounds, or by direct enzyme activity assays or binding assays provided that the assay can detect weak binders (Kd or IC₅₀ of about 1 mM). Methods of soaking crystals in mixtures are known to those of ordinary skill in the art and are discussed in, for example, Nienaber et al., U.S. Pat. No. 6,297,021, issued Oct. 2, 2001. The combination of core fragments or compounds included in mixtures may, for example, be designed to make it easier to differentiate the fragments or compounds when viewing the electron density of the structure. In one example, about half of the fragments or compounds in the mixture comprise a substituent having anomalous dispersion properties, such as, for example, bromine. The crystals are then subjected to X-ray crystallographic structure determination. Molecules that have desirable binding properties are then selected for further elaboration, either through a second round of linear library synthesis, or by building out the handle by adding additional functional groups, or through the synthesis of combinatorial libraries.

Those of ordinary skill in the art recognize that although the number of compounds is not limited to that presented in this example, the goal is to create libraries with a limited, selected, number of compounds. This contrasts with the thousands to hundreds of thousands of compounds that may be used in a traditional initial screen.

The information obtained in the first steps may then be used to further refine the substitutions on each central core, for further exploration at each handle with selected libraries. Or, this information may be used to expand the analysis to include a much larger group of compounds, including all permutations of the various groups at each handle. For example, compounds selected after a first round of linear library screening may then be used as the base of a new linear library, which will then be screened. Or, a selected compound may be the base of a more traditional combinatorial library, which is then screened. A selected compound may also be used for computational drug design, with specific changes made to portions of the compound to improve its contact with a binding site. Computational chemistry software may be used to design, dock, and select compounds for further analysis. These compounds are then subjected to a next cycle of assays and structural analysis.

In one aspect of the invention, once a desired structure-activity-relationship, or SAR, is obtained, a combinatorial library may be developed for further activity optimization. A combinatorial library may be designed, for example, where an about five fold, about ten fold, about twenty fold, about one hundred fold or greater increase in activity of a compound of a core fragment library or linear library as compared to the screening results of the preceding library. This combinatorial library may include changes at more than one handle at a time; it may combine particular handles identified on separate compounds in the linear library. The structural information obtained in the earlier steps can help to direct the design of the combinatorial library. In some aspects, where sufficient information and activity is obtained from the core fragment library screening, a combinatorial library may be prepared and screened directly after the core fragment library screening.

As a core fragment is further elaborated, it may become larger. The molecular weight of an elaborated fragment, or lead candidate, may be, for example less than about 500, less than about 450, less than about 400, less than about 350, or less than about 300 daltons.

### EXAMPLE 4

### Computational Screening of Core Fragment-Handle Combinations for Synthesis of Linear Libraries

To computationally select handle modifications, various methods may be used. In one example, each potential reagent out of a pool of potential reagents compatible with a given handle, for example about 10,000 reagents, may be used to generate a virtual linear library in silico. To screen the linear library, energetically favorable conformers are generated for each derivative of the virtual library. Each conformer is placed in the crystallographically determined core fragment position in the desired protein binding site, and subjected to energy minimization. Unfavorable conformations are removed and top scoring substituents are selected using the MM/PBSA binding free energy method. (P. A. Kollman, et al., Calculating Structures and Free Energies of Complex Molecules: Combining Molecular Mechanics and Continuum Models. Accts. Chem. Res. 33, 889-897 (2000)).

In one example of the present invention, once a core fragment is selected, it can be subjected to an in silico reaction to generate one virtual library per handle Sterically accessible and/or energetically favorable conformers are generated, using software such as, for example, OMEGA (OpenEye), Catalyst (Accelrys), MOE (CCG) and SYBYL (Tripos). in the crystallographically determined core fragment position using, for example MOE (CCG) and DOCK. The conformer/binding site combination is subjected to energy minimization using, for example InsightII (Accelrys), MOE (CCG) SYBYL (Tripos) and AMBER, and unfavorable conformations, such as, for example, those that have high intramolecular energy, such as, for example, those that have an intramolecular energy greater than about 5.0 kcal/mol, are removed. The top scoring substituents from the remaining conformations are selected with MM/PBSA and synthesized for further analysis.

Other computational chemistry methods may be used to select components of a linear or combinatorial library. These programs may also be used to design modifications to a compound, such as a core fragment, to obtain a lead candidate.

Computer modeling techniques may be used to assess the potential modulating or binding effect of a chemical compound on target protein. If computer modeling indicates a strong interaction, the molecule may then be synthesized and tested for its ability to bind to target protein and affect (by inhibiting or activating) its activity.

Modulating, for example, compounds that inhibit or activate a biological target molecule activity, or other binding compounds of target protein may be computationally evaluated and designed by means of a series of steps in which chemical groups or fragments are screened and selected for their ability to associate with the individual binding pockets or other areas of a target protein. Several methods are available to screen chemical groups or fragments for their ability to associate with target protein. This process may begin by visual inspection of, for example, the active site on the computer screen based on the target protein coordinates. Selected fragments or chemical groups may then be positioned in a variety of orientations, or docked, within an individual binding pocket of target protein (Blaney, J. M. and Dixon, J. S., Perspectives in Drug Discovery and Design, 1 :301, 1993). Manual docking may be accomplished using software such as Insight II (Accelrys, San Diego, Calif.) MOE (CCG); and SYBYL (Molecular Modeling Software, Tripos Associates, Inc., St. Louis, Mo., 1992), followed by energy minimization and molecular dynamics with standard molecular mechanics force fields, such as CHARMM (Brooks, et al:, J. Comp. Chem. 4:187-217, 1983). More automated docking may be accomplished by using programs such as DOCK (Kuntz et al., J. Mol. Biol., 161:269-88, 1982; DOCK is available from University of California, San Francisco, Calif.); AUTODOCK (Goodsell & Olsen, Proteins: Structure, Function, and Genetics 8:195-202, 1990; AUTODOCK is available from Scripps Research Institute, La Jolla, Calif.); GOLD (Cambridge Crystallographic Data Centre (CCDC); Jones et al., J. Mol. Biol. 245:43-53, 1995); and FLEXX (Tripos, St. Louis, Mo.; Rarey, M:, et al., J. Mol. Biol. 261:470-89, 1996); AMBER (Weiner, et al., J. Am. Chem. Soc. 106: 765-84, 1984) and C² MMFF (Merck Molecular Force Field; Accelrys, San Diego, Calif.). Other approriate programs are described in, for example, Halperin, et al.

Specialized computer programs may also assist in the process of selecting fragments or chemical groups. These include DOCK; GOLD; LUDI; FLEXX (Tripos, St. Louis, Mo.; Rarey, M., et al., J. Mol. Biol. 261:470-89, 1996); and GLIDE (Eldridge, et al., J. Comput. Aided Mol. Des. 11:425-45, 1997; Schrodinger, Inc., New York). Other appropriate programs are described in, for example, Halperin, et al.

Other molecular modeling techniques may also be employed in accordance with this invention. See, e.g., Cohen et al., J. Med. Chem. 33:883-94, 1990. See also, Navia & Murcko, Current Opinions in Structural Biology 2:202-10, 1992; Balbes et al., Reviews in Computational Chemistry, 5:337-80, 1994, (Lipkowitz and Boyd, Eds.) (VCH, N.Y.); Guida, Curr. Opin. Struct. Biol. 4:777-81, 1994. During design and selection of compounds by the above methods, the efficiency with which that compound may bind to target protein may be tested and optimized by computational evaluation. For example, a compound that has been designed or selected to function as a target protein inhibitor may occupy a volume not overlapping the volume occupied by the active site residues when the native substrate is bound, however, those of ordinary skill in the art will recognize that there is some flexibility, allowing for rearrangement of the main chains and the side chains. In addition, one of ordinary skill may design compounds that could exploit protein rearrangement upon binding, such as, for example, resulting in an induced fit. An effective target protein inhibitor must preferably demonstrate a relatively small difference in energy between its bound and free states (i.e., it must have a small deformation energy of binding and/or low conformational strain upon binding). Thus, the most efficient target protein inhibitors should, for example, be designed with a deformation energy of binding of not greater than about 10 kcal/mol, for example, not greater than about 7 kcal/mol, for example, not greater than about 5 kcal/mol and, for example, not greater than about 2 kcal/mol. Target protein inhibitors may interact with the protein in more than one conformation that is similar in overall binding energy. In those cases, the deformation energy of binding is taken to be the difference between the energy of the free compound and the average energy of the conformations observed when the inhibitor binds to the enzyme. Methods of calculating energies are known to those of ordinary skill in the art and include, for example, MOE v2004.03 from Chemical Computing Group using MMFF94, or Open Eye software using MMFF94s. MMFF94 and MMFF94s (Merck Molecular Mechanics Force Field) are discussed in, for example, Halgren, J. Comput. Chem., 17, 490-519 (1996); Halgren, J. Comput. Chem., 17, 520-552 (1996); Halgren, J. Comput. Chem., 17, 553-586 (1996); Halgren and Nachbar, J. Comput. Chem., 17, 587-615 (1996); Halgren, J. Comput. Chem., 17, 616-641 (1996); Halgren, J. Comput. Chem., 20, 720-729 (1999); and Halgren, J. Comput. Chem., 20, 730-748 (1999).

A compound selected or designed for binding to a target protein may be further computationally optimized so that in its bound state it would, for example, lack repulsive electrostatic interaction with the target protein. Non-complementary electrostatic interactions include repulsive charge-charge, dipole-dipole and charge-dipole interactions. Specifically, the sum of all electrostatic interactions between the inhibitor and the protein when the inhibitor is bound to it may make a neutral or favorable contribution to the enthalpy of binding.

Specific computer software is available in the art to evaluate compound deformation energy and electrostatic interaction. Examples of programs designed for such uses include: Gaussian 94, revision C (Frisch, Gaussian, Inc., Pittsburgh, Pa. ©1995); AMBER, version 7 (Kollman, University of California at San Francisco, ©2002); QUANTA/CHARMM (Accelrys, Inc., San Diego, Calif., ©1995); Insight II/Discover (Accelrys, Inc., San Diego, Calif., ©1995); DelPhi (Accelrys, Inc., San Diego, Calif., ©1995); and AMSOL (University of Minnesota). These programs may be implemented, for instance, using a computer workstation, as are well known in the art, for example, a LINUX, SGI or Sun workstation. Other hardware systems and software packages will be known to those skilled in the art.

General synthetic methods for synthesizing core fragment/handle combinations of the present invention may be found in, for example, U.S. Pat. No. 5,756,466 .

### EXAMPLE 5

### Design of a Drug Candidate

The methods of the present invention may be used, for example, in the design of a drug candidate using the steps presented in this Example. Those of ordinary skill in the art may perform the methods outlined in these steps, modify the order or timing of these steps, as well as add additional steps, according to the methods of the present invention.

1. select a core fragment library from commercially available or custom synthesized molecules

2. screen the core fragment library biochemically, biophysically, and/or crystallographically for hits against a target biomolecule

3. computationally elaborate hits into all readily synthesized analogs at each handle, 1 library per handle

4. score the virtual library computationally with MM/PBSA

5. select top-scoring compounds for synthesis

6. obtain compounds selected in step 5

7. assay compounds of step 6 biochemically

8. solve crystal structures of selected active compounds from step 7 in association with the target biomolecule

9. design a linear or combinatorial library using several of the most active substituents from each linear library

10. obtain compounds designed in step 9

11. assay compounds of step 10 biochemically

12. solve crystal structures of selected active compounds from step 11 in association with the target biomolecule

13. continue iterating steps 3-12 until desired activity is reached.

### EXAMPLE 6

### Design of a SYK Inhibitor

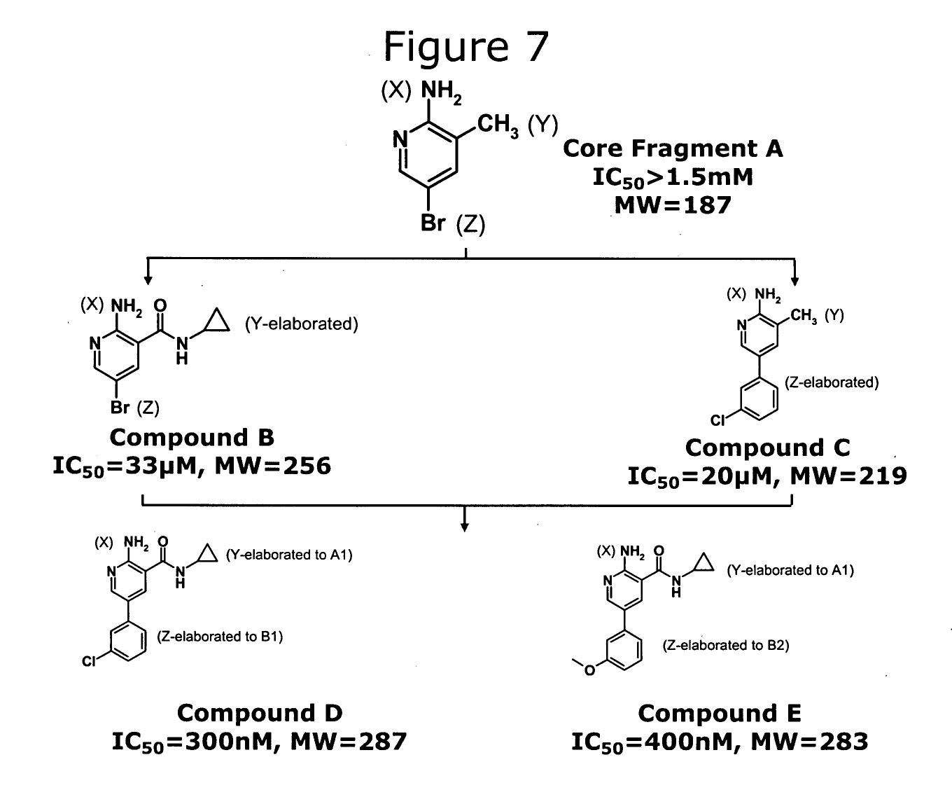

The present invention may be used to design and identify a potent compound having activity against a biological target molecule. In one aspect of the invention, a core fragment may be screened against one target protein, and, as the core fragment is developed through elaboration at one or more of the handles, the resulting elaborated compound may be screened against another target protein, as a non-limiting example, a protein that is a member of the same protein family as the first target protein. In the exemplary case. of a target protein with enzymatic activity, the other target may be an enzymatic protein with the same or overlapping enzyme classification (EC) as provided by the Nomenclature Committee of the International Union of Biochemistry and Molecular Biology (NC-IUBMB) in consultation with the IUPAC-IUBMB Joint Commission on Biochemical Nomenclature (JCBN). The following is an example of how the methods of the present invention may be applied where one target protein is used in the development of a compound having activity against a second target protein, as depicted in FIG. 7. Those of ordinary skill in the art would readily understand that these methods may be modified for the use of a single target protein, or even more than two target proteins. In one such method, the same target protein is used for the initial screening and through development of the molecule.

A core fragment library comprising core fragments comprising substituents having anomalous dispersion properties is obtained. Core fragments may be synthesized using methods known to ordinary skill in the art, or acquired from commercial suppliers, such as, for example, SIGMA-ALDRICH, LANCASTER, FLUKA, ACROS, MAYBRIDGE, and CHEMBRIDGE. Crystals of the kinase domain of PAK4 are obtained as in, for example, U.S. Ser. No. 10/406,676, filed Apr. 2, 2003, Crystals and structures of PAK4KD Kinase PAK4KD, Antonysamy, et al. (US-2003-0229453-A1), hereby incorporated by reference herein in its entirety. Crystals are soaked in solutions of five core-fragment mixture with 10 mM sample concentration for each fragment in the soaking mixture solutions and crystals are then isolated from the soaking solution and subjected to structure determination according to the methods presented in U.S. Ser. No. 10/406,676, filed Apr. 2, 2003, Crystals and structures of PAK4KD Kinase PAK4KD, Antonysamy, et al. (US-2003-0229453-A1). The protein structure solution reveals core fragments that associate with a binding site on Pak4. One such fragment is fragment A. The core fragments are also screened against Pak4 for biochemical activity by using a Pak4 PK-LDH coupled assay such as, for example, assays presented in U.S. Ser. No. 10/406,676, above. The biochemical activity of core fragment A against Pak4 is IC₅₀>1.5 mM.

Core fragment A is selected for further elaboration into a linear library. First, handles which are going to be elaborated are selected based on the X-ray complex structure of the protein and the core fragments. The Pak4 protein structure with core fragment A in its binding site shows that handle X makes direct and specific interaction with Pak4. Hence, only handles Y and Z are selected for further elaboration. Each handle is elaborated individually into a virtual library by generating all possible synthetic derivatives using compatible, commercially available reagents. Elaborated handles, or derived substituents, may be designed by modification of, substitution of, or addition to, a handle. For example, the aromatic methyl group of latent handle Y can be oxidized to a carboxylic acid and then coupled with amines to form amides. For example, the aromatic bromine of latent handle Z can be used to perform Suzuki couplings with boronic acid reagents. Core fragment A is then elaborated by the design of linear libraries, having modifications at the two different core fragment A handles, Y and Z, by using computational screening techniques of central core-handle combinations, as set forth, for example, herein in Example 3, and linear libraries are synthesized. The size of a linear library may be, for example, 10-50 compounds. The linear library compounds are screened against target proteins, for example, Pak4 and Syk for biochemical activity, and then all or some selected compounds are used in soaking experiments, either singly, or in mixtures, with crystals of Syk KD. Compounds having biochemical activity may be chosen for the structural experiments, as well as, for example, compounds not having biochemical activity, as both may yield information helpful for the next design steps. The linear library compounds, and core fragment A, are also used in SYK activity assays by using a SYK PK-LDH coupled assay. Two compounds, from two different linear libraries, compounds B, from a linear library having modifications at handle Y, and C, from a linear library having modifications at handle Z, are examples identified as having increased activity against SYK as compared to core fragment A. Information obtained from the structures of compounds B,C and other linear library compounds are then used to design a combinatorial library, comprising compounds having modifications at one, or more than one, handles by using computational screening technique of central core-handle combinations-and a combinatorial library is synthesized. The size of the combinatorial library may be, for example, 10-50 compounds. The compounds of the combinatorial library are then screened against Syk for biochemical activity, and then all or some selected compounds are used in soaking experiments with crystals of Syk Kd. In the present case, Compound D is an example of a compound designed to include both of the elaborated handles from compounds B (handle Y) and C (handle Z), and Compound E comprises the same elaborated handle Y of compound B, but a modified handle Z when compared to compound B. Compounds D and E are examples of lead candidates that may be designed and identified using methods of the present invention. Compounds D and E may be, for example, further elaborated through the design of additional linear or combinatorial libraries, or the structures of Compounds D and E in association with SYK may be used to design compounds having improved binding to the SYK binding site, which are also tested for biochemical activity. Lead candidates may be tested in cells or animals and further elaborated to have improved solubility or ADMET properties, as needed.

### EXAMPLE 6.1

### Compound Synthesis

Compounds of this example, and FIG. 7, may be synthesized, for example, as presented herein.

SYK inhibitor compounds presented in the Examples section and FIG. 7 of the present application may be prepared, for example, using the following methods.

Methods of Preparation of End Products.

General Scheme

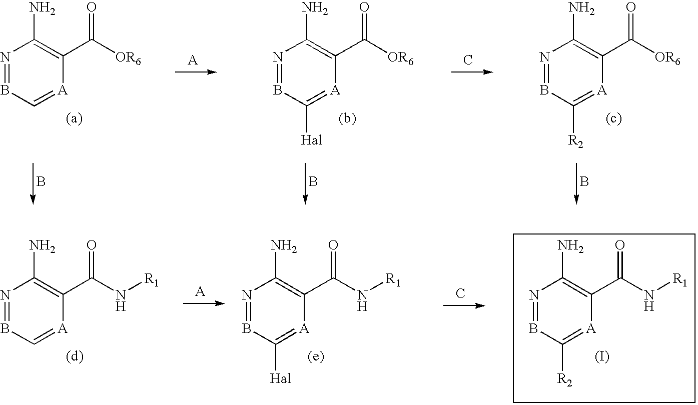

These methods are comprised of:

A:

Synthesis of the required halogenated intermediates (b) or (e) by reacting a derivative (a) either as the free acid (R₆=H) or as an ester, or (d) with a suitable halogenating reagent, such as bromine or iodine or a suitable halogen containing reagents such as ICl, N-bromosuccinimide or N-iodosuccinimide in suitable solvents such as acetic acid, DMF or methylene chloride at temperatures ranging from 20° C. to 100° C.

B:

Synthesis of the required amide intermediates (d) and (e) and amide formation to obtain compounds of the general formula I, wherein A, B, R₁ and R₂ are as defined in formula I by activating the carboxylic acid by use of reagents such as oxalyl chloride, thionyl chloride, O-(benzotriazol-1-yl)-1,1,3,3-tetramethyluronium hexafluorophosphate, benzotriazol-1-yloxy-tris(pyrrolidino)phosphonium hexafluorophosphate, carbonyldiimidazole, dicyclohexylcarbonyldiimide or 1-[3-(dimethylamino)propyl]-3-ethylcarbodiimide and subsequent treatment with an amine R₁NH₂ either in the presence of additives such as 4-dimethylaminopyridine, hydroxybenzotriazole, hydroxy-7-azabenzotriazole or without.

An alternative procedure to obtain amide intermediates (d) and (e) and compounds of the general formula I, wherein A, B, R₁ and R₂ are as defined in formula I consists of treatment of the corresponding esters (a), (b) or (c) with an amine in solvents such as, but not limited to DMSO, DMF, DMA, NMP, ethanol, butanol or pentanol either directly or in the presence of suitable reagents or catalysts such as scandium trifluoromethanesulfonate, ytterbium trifluoromethanesulfonate, trimethylaluminum or boron trifluoride at temperatures ranging from 20° C. to 250° C. using either conventional heating or microwave irradiation.

C:

Procedures to furnish intermediates (c) or compounds of the general formula I, wherein A, B, R₁ and R₂ are as defined in formula I by coupling of halogen containing intermediates (b) and (e) with

a) boronic acids or esters in the presence of suitable metal catalysts such as palladium on charcoal, tetrakis(triphenylphosphino)palladium(0), [1,1′-bis(diphenylphosphino)-ferrocene]palladium(II)-dichloride, tris(dibenzylideneacetone)dipalladium(0) and additives such as, but not limited to, triphenylphosphine, tris-tert-butylphosphine, 2-(biphenyl)dicyclohexylphosphane, tris(ortho-tolyl)phosphine, cesium fluoride, cesium carbonate, sodium carbonate, sodium bicarbonate, sodium hydroxide, potassium carbonate, potassium fluoride in solvents such as ethylene glycol dimethyl ether, water, ethanol, dioxane, toluene, xylene and mixtures thereof at temperatures ranging from 20° C. to 200° C. using either conventional heating or microwave irradiation.

b) aryl stannanes in the presence of suitable metal catalysts such as palladium on charcoal, lithium tetrachloropalladate, tetrakis(triphenylphosphino)palladium(0), [1,1′-bis(diphenylphosphino)ferrocene]palladium(II)-dichloride, palladium(II)-acetate, tris(dibenzylideneacetone)dipalladium(0) and additives such as, but not limited to, triphenylphosphine, tris-tert-butylphosphine, 2-(biphenyl)dicyclohexylphosphane, tris(ortho-tolyl)phosphine, tris(2-furyl)phosphine, triphenylarsine, cesium fluoride, potassium fluoride, copper(I)-oxide, silver(I)-oxide and lithium chloride in solvents such as ethylene glycol dimethyl ether, water, dioxane, toluene, xylene, ortho-dichlorobenzene and mixtures thereof at temperatures ranging from 20° C. to 200° C. using either conventional heating or microwave irradiation.

c) electron deficient olefins such as methyl acrylate in the presence of suitable metal catalysts such as palladium(II)-acetate and additives such as, but not limited to, triphenylphosphine, tris(ortho-tolyl)phosphine, and triethyl amine in solvents such as DMF or DMA at temperatures ranging from 20° C. to 200° C. using either conventional heating or microwave irradiation.

The compounds of the examples and FIG. 7 may be made by the procedures and techniques above, as well as by known organic synthesis techniques, including the techniques disclosed in Littke, A. F., et al., J. Am. Chem. Soc., 122: 4020-4028 (2000) but utilizing 3-amino-6-iodo-pyrazine-2-carboxylic acid derivatives for coupling with boronic acids (illustrated in Scheme 4), which reference is incorporated herein in its entirety. The compounds may be made by the following general reaction schemes.

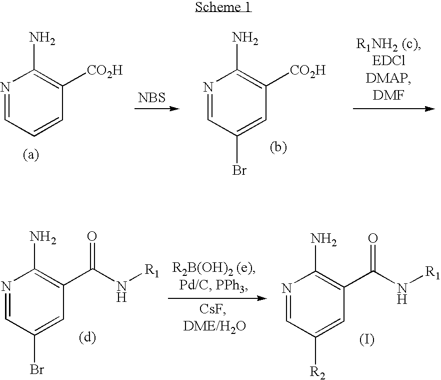

In the above reaction Scheme 1, 2-aminonicotinic acid (a), upon treatment with N-bromosuccinimide (NBS) provides the brominated pyridine (b), which is then treated with amine (c), 4-dimethyl-aminopyridine (DMAP) and 1-(3-dimethylaminopropyl)-3-ethylcarbodiimide hydrochloride (EDCI) to yield amide (d). Subsequent treatment of (d) with boronic acid (e), palladium on carbon (Pd/C), triphenylphosphine (PPh₃) and cesium fluoride affords compounds of structure (I) where A is CH, B is CH, X is O, and Y is NH, and R₂ is aryl. The general reaction illustrated in Scheme 1 is also applicable for the synthesis of compounds of structure (I) where A is CH, B is CH, X is O, R₂ is aryl, and Y is NR4, by utilizing R₁R₄NH in place of R₁NH₂ (c).

In an alternative method, as shown in Scheme 2, treatment of (d) with boronic acid (e), dichloro[1,1′-bis(diphenylphosphino)ferrocene]palladium(II) dichloromethane adduct [PdCl₂(dppf)] and cesium fluoride provides compounds of structure (1) where A is CH, B is CH, X is O, Y is NH, and R₂ is aryl. The general reaction illustrated in Scheme 2 is also applicable for the synthesis of compounds of structure (I) where A is CH, B is CH, X is O, R₂ is aryl, and Y is NR4, by utilizing R₁R₄NH in place of R₁NH₂ (c).

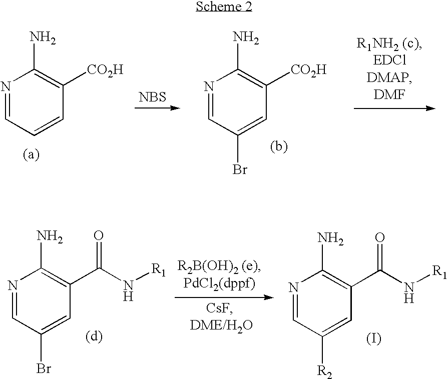

In reaction Scheme 3, 2-aminopyrazine-3-carboxylic acid methyl ester (a), upon treatment with N-iodosuccinimide (NIS) affords the iodinated pyrazine (b), which is then treated with boronic acid (c) in the presence of tris(dibenzylideneacetone)dipalladium(0) [Pd₂(dba)₃], tris-tert-butylphosphine and potassium fluoride to yield the coupled pyrazine product (d). Subsequent treatment of (d) with amine (e) in the presence of scandium trifluoromethanesulfonate in DMSO affords compounds of structure (I) where A is N, B is CH, X is O, Y is NH, and R₂ is aryl.

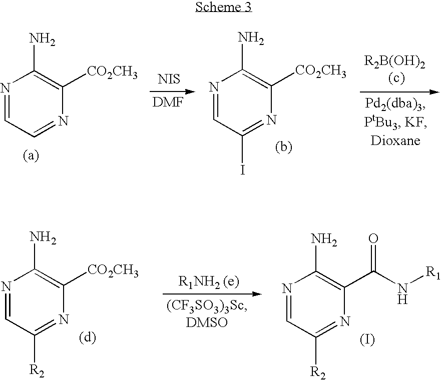

In an alternative method, shown in Scheme 4, saponification of (d) with sodium hydroxide in ethanol-water affords the corresponding acid, which upon treatment with amine R₁NH₂ (f) and (benzotriazol-1-yloxy)tripyrrolidinophosphonium hexafluorophosphate [PyBOP] in DMF compounds of structure (I) where A is N, B is CH, X is O, Y is NH, and R₂ is aryl.

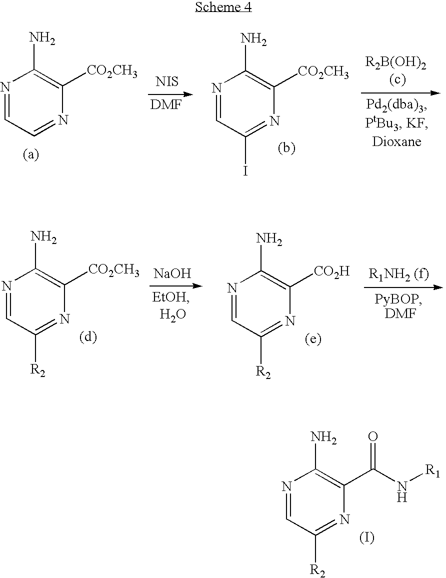

In reaction Scheme 5, 3-aminopyridazine-4-carboxylic acid (a) [J. Org. Chem. 1985, 50, 346-350], upon treatment with N-iodosuccinimide (NIS) affords the iodinated pyridazine (b), which is then treated with amine (c), 1-hydroxy-benzotriazole (HOBT) and 1-(3-dimethylaminopropyl)-3-ethylcarbodiimide hydrochloride (EDCI) to yield amide (d). Subsequent treatment of (d) with boronic acid (e), a suitable palladium catalyst system such as palladium on charcoal with triphenylphosphine, or dichloro[1,1′-bis(diphenyl phosphino)ferrocene]palladium(II) dichloromethane adduct, and cesium fluoride affords compounds of structure (I) where A is CH, B is N, X is O, Y is NH, and R₂ is aryl. The general reaction illustrated in Scheme 5 is also applicable for the synthesis of compounds of structure (I) where A is CH, B is N, X is O, R₂ is aryl and Y is NR4, by utilizing R₁R₄NH in place of R₁NH₂ (e).

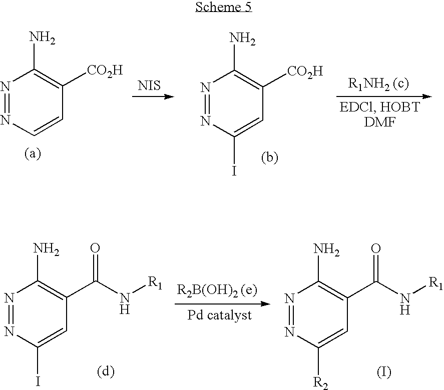

In reaction Scheme 6, 5-bromo 2-aminonicotinic acid (a) is treated with methyl acrylate (b) and triethylamine in the presence of palladium(II)-acetate and triphenylphosphine to yield the coupled olefin (I) where A is CH, B is CH, X is O, Y is OH, and R₂ is CHCHCO₂CH₃.

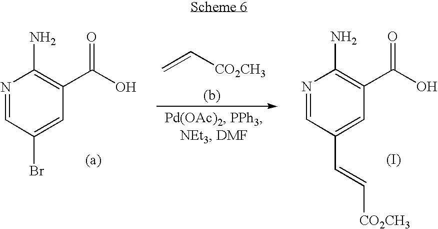

In Scheme 7, 2-amino-5-bromonicotinic acid, synthesized as described in Scheme 1, is reacted with a boronic acid (b) in the presence of tetrakis(triphenylphosphino)palladium(0) and cesium carbonate. The general reaction illustrated in Scheme 7 is applicable for the synthesis of compounds of structure (I) where where A is CH, B is CH, X is O, Y is OH and R₂ is aryl.

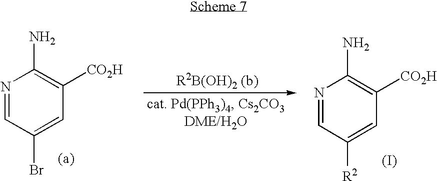

### Synthesis of 5-Bromo-2-aminonicotinic Acid

To a stirring solution of 2-aminonicotinic acid (25 g, 0.181 mol) in DMF (500 ml) at room temperature was added N-bromosuccinimide (33.5 g). After 4 hours, the reaction was quenched with water. The brown solid was filtered and washed with water several times. The resulting solid was dissolved in methanol and treated with activated carbon. The mixture was filtered and the solution was concentrated to give 11.5 g of 5-bromo-2-aminonicotinic acid as a pale yellow solid (11.2 g, 29% yield); ¹H-NMR (d₆-DMSO) δ

d: 13.3 (broad s, 1H, COOH), 8.24 (s, 1H), 8.08 (s, 1H), 7.35 (broad s, 2H, NH₂); HPLC/MS m/z: 218 [MH]⁺.

### Synthesis of 2-Amino-5-bromo-N-cyclopropyl-nicotinamide

To a solution of 5-bromo-2-aminonicotinic acid (0.50 g, 2.30 mmol), EDCI (0.68 g, 3.45 mmol) and DMAP (0.56 g, 4.60 mmol) in DMF (6 ml) was added cyclopropylamine (0.19 g, 3.45 mmol). The reaction was stirred overnight at room temperature, before quenching with water. The precipitate was filtered, washed with water, and dried in vacuo which afforded the title compound as a yellow solid (0.406 g, 69% yield). ¹H-NMR (d₆-DMSO) d: 8.47 (s, 1H), 8.12 (s, 1H), 8.02 (s, 1H), 7.24 (broad s, 2H), 2.78 (m, 1H), 0.68 (m, 2H), 0.54 (m, 2H); HPLC/MS m/z: 257 [MH]⁺.

### Synthesis of 2-Amino-5-(3-chloro-phenyl)-N-cyclopropyl-nicotinamide

To 2-amino-5-bromo-N-cyclopropyl-nicotinamide (51 mg, 0.2 mmol), Pd/C (10%w., 10 mg, 5 mol %), PPh₃ (10.5 mg, 20 mol %), and 3-chlorophenylboronic acid (37 mg, 0.24 mmol) in a Smith process vial was added DME (1 ml), water (0.6 ml), and 4N aqueous solution of CsF (0.4 ml). The reaction was run in a Personal Chemistry SmithCreator microwave at 160° C. for 1200s. The reaction mixture was then extracted with ethyl acetate, and the combined extracts were washed with 1N NaOH solution, then water. The organic layer was dried over anhydrous sodium sulfate, filtered, and concentrated in vacuo. Purification on silica gel with a gradient of ethyl acetate/hexane afforded the title compound as a solid (19 mg, 33% yield). ¹H-NMR (d₆-DMSO) d: 8.55 (broad d, 1H, NH), 8.44 (d, 1H), 8.14 (d, 1H), 7.76 (d, 1H), 7.63 (m, 1H), 7.45 (t, 1H), 7.34 (m, 1H), 7.29 (broad s, 2H), 2.80 (m 1H), 0.71 (m, 2H), 0.58 (m, 2H); HPLC/MS m/z: 288 [MH]⁺.

### Synthesis of 2-Amino-5-(3-benzyloxy-phenyl)-N-cyclopropyl-nicotinamide

To 2-amino-5-bromo-N-cyclopropyl-nicotinamide (51 mg, 0.2 mmol), 3-benzyloxyphenylboronic acid (55 mg, 0.24 mmol) and PdCl₂(dppf)·CH₂Cl₂ (29 mg, 0.04 mmol) in a Smith process vial was added DME (1 ml), water (0.6 ml), and 4N aqueous solution of CsF (0.4 mL). The reaction was run in a Personal Chemistry SmithCreator microwave at 160° C. for 1200 s. The reaction mixture was then extracted with ethyl acetate, and the combined extracts were washed with 1N NaOH solution, then water. The organic layer was dried over anhydrous sodium sulfate, filtered, and concentrated in vacuo. Purification on silica gel with a gradient of ethyl acetate/hexane afforded pure title compound as an off-white solid (45 mg, 63% yield); ¹H-NMR (d₆-DMSO) d: 8.38 (d, 1H), 7.65 (d, 1H), 7.35 (m, 6H), 7.07 (m, 2H), 6.94 (m, 1H), 6.43 (broad s, 2H), 6.24 (broad s, 1H), 5.11 (s, 2H), 2.87 (m, 1H), 0.88 (m, 2H), 0.63 (m, 2H); HPLC/MS m/z: 360 [MH]⁺; mp. 177-178° C.

### EXAMPLE 7

### Expression of Target Protein

Target protein, including polypeptides, may be chemically synthesized in whole or part using techniques that are well known in the art (see, e.g., Creighton, Proteins: Structures and Molecular Principles, W. H. Freeman & Co., NY, 1983). For purposes of these examples, the terms “protein” and “polypeptide” are interchangeable.

Gene expression systems may be used for the synthesis of target polypeptides. Expression vectors containing the polypeptide coding sequence and appropriate transcriptional/translational control signals, that are known to those skilled in the art may be constructed. These methods include in vitro recombinant DNA techniques, synthetic techniques and in vivo recombination/genetic recombination. See, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989.

Host-expression vector systems may be used to express target protein. These include, but are not limited to, microorganisms such as bacteria transformed with recombinant bacteriophage DNA, plasmid DNA or cosmid DNA expression vectors containing the target protein coding sequence; yeast transformed with recombinant yeast expression vectors containing the target protein coding sequence; insect cell systems infected with recombinant virus expression vectors (e.g., baculovirus) containing the target protein coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the target protein coding sequence; or animal cell systems. The protein may also be expressed in human gene therapy systems, including, for example, expressing the protein to augment the amount of the protein in an individual, or to express an engineered therapeutic protein. The expression elements of these systems vary in their strength and specificities.

Specifically designed vectors allow the shuttling of DNA between hosts such as bacteria-yeast or bacteria-animal cells. An appropriately constructed expression vector may contain: an origin of replication for autonomous replication in host cells, one or more selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency.

The expression vector may also comprise various elements that affect transcription and translation, including, for example, constitutive and inducible promoters. These elements are often host and/or vector dependent. For example, when cloning in bacterial systems, inducible promoters such as the T7 promoter, pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, mammalian promoters (e.g., metallothionein promoter) or mammalian viral promoters, (e.g., adenovirus late promoter; vaccinia virus 7.5K promoter; SV40 promoter; bovine papilloma virus promoter; and Epstein-Barr virus promoter) may be used.

Various methods may be used to introduce the vector into host cells, for example, transformation, transfection, infection, protoplast fusion, and electroporation. The expression vector-containing cells are clonally propagated and individually analyzed to determine whether they produce target protein. Various selection methods, including, for example, antibiotic resistance, may be used to identify host cells that have been transformed. Identification of target polypeptide-expressing host cell clones may be done by several means, including but not limited to immunological reactivity with anti-target protein antibodies, and the presence of host cell-associated target protein activity.

Expression of target protein cDNA may also be performed using in vitro produced synthetic mRNA. Synthetic mRNA can be efficiently translated in various cell-free systems, including but not limited to wheat germ extracts and reticulocyte extracts, as well as efficiently translated in cell-based systems, including, but not limited, to microinjection into frog oocytes.

To determine the target protein cDNA sequence(s) that yields optimal levels of target protein activity and/or target protein protein, modified target protein cDNA molecules are constructed. A non-limiting example of a modified cDNA is where the codon usage in the cDNA has been optimized for the host cell in which the cDNA will be expressed. Host cells are transformed with the cDNA molecules and the levels of target protein RNA and/or protein are measured.

Levels of target protein in host cells are quantitated by a variety of methods such as immunoaffinity and/or ligand affinity techniques-specific affinity beads or target protein-specific antibodies are used to isolate ³⁵S-methionine labeled or unlabeled target protein. Labeled or unlabeled target protein is analyzed by SDS-PAGE. Unlabeled target protein is detected by Western blotting, ELISA or RIA employing target protein-specific antibodies.

Following expression of target protein in a recombinant host cell target protein may be recovered to provide target protein in active form. Several target protein purification procedures are available and suitable for use. Recombinant target protein may be purified from cell lysates or from conditioned culture media, by various combinations of, or individual application of, fractionation, or chromatography steps that are known in the art.

In addition, recombinant target protein can be separated from other cellular proteins by use of an immuno-affinity column made with monoclonal or polyclonal antibodies specific for full length nascent target protein or polypeptide fragments thereof. Other affinity based purification techniques known in the art may also be used.

Alternatively, target protein may be recovered from a host cell in an unfolded, inactive form, e.g., from inclusion bodies of bacteria. Proteins recovered in this form may be solubilized using a denaturant, e.g., guanidinium hydrochloride, and then refolded into an active form using methods known to those skilled in the art, such as dialysis.

### EXAMPLE 7.1

### Expression of SYK Kinase Domain

Human liver cDNA was synthesized using a standard cDNA synthesis kit following the manufacturers' instructions. The template for the cDNA synthesis was mRNA isolated from Hep G2 cells [ATCC HB-8065] using a standard RNA isolation kit. An open-reading frame for SYKKD was amplified from the human liver cDNA by the polymerase chain reaction (PCR) using the following primers:

| | |
|---|---|
| Forward primer: | GAGGAGATCAGGCCCAAG |
| Reverse primer: | CGTTCACCACGTCATAGTAG |

The PCR product (840 base pairs expected) was electrophoresed on a 1.2% E-gel (Cat. #G5018-01, Invitrogen Corporation) and the appropriate size band was excised from the gel and eluted using a standard gel extraction kit. The eluted DNA was TOPO ligated into a GATEWAY™ (Invitrogen Corporation) adapted pcDNA6 AttB HisC vector which was custom TOPO adapted by Invitrogen Corporation. The resulting sequence of the gene after being TOPO ligated into the vector, from the start sequence through the stop site was as follows: ATG GCC CTT 3′[SYK]KD5′AA GGG CAT CAT CAC CAT CAC CAC TGA The SYKKD expressed using this vector has an N-terminal methionine, the kinase domain of SYKKD, and a C terminal 6× His-tag.

Plasmids containing TOPO ligated inserts were transformed into chemically competent TOP 10 cells (Invitrogen Corporation, Cat.#C4040-10). Colonies were then screened for inserts in the correct orientation and small DNA amounts were purified using a “miniprep” procedure from 2ml cultures, using a standard kit, following the manufacturer's instructions. For standard molecular biology protocols followed here, see also, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989. The DNA that was in the “correct” orientation was then sequence verified.

A standard GATEWAY™ BP recombination was performed into pDONR201 (Invitrogen Corporation, Cat.#11798014. Gateway technology Cat.#11821014) and the recombination reaction was transformed into chemically competent TOP 10 cells (Invitrogen Corporation, Cat.#C4040-10), and plated on selective media. One colony was picked into a miniprep and DNA was obtained (the “entry vector”).

The “entry vector” DNA is used in a standard GATEWAY™ LR recombination with pDEST8™ ( Invitrogen Corporation, Cat.#11804010) and transformed into chemically competent TOP 10 cells (Invitrogen Corporation, Cat.#C4040-10), and plated on selective media. One colony was picked into a miniprep and DNA was obtained (the “destination vector”).

The “destination vector” was then transformed into DH10 BAC chemically competent cells (Invitrogen Corporation, Cat#10361012) which uses site specific transposition to insert a foreign gene into a bacmid propogated in E.coli. The transformation was then plated on selective media. 1-2 colonies were picked into minipreps. The Nautilus Genomic miniprep kit (Active Motif, Cat.#50050) was used to purify the bacmid DNA. The bacmid was then verified by PCR.

The bacmid was transfected and expressed in SF9 cells using the following standard Bac to Bac protocol (Invitrogen Corporation, Cat.#10359-016)

Day 0

Seeded 9×10E5 cells per 35 mm well (of a 6 well plate) in 2 ml Sf-900II SFM (Cat. #10902-104, Invitrogen Corporation) containing 1% Penicillin/Streptomycin (Cat. # 15140122, Invitrogen Corporation).

Allowed cells to attach at 27° C. for 1 hour

In a Falcon 2059 polypropylene 12×75 mm tube prepared the following solutions.

1. Dilute 5 μl of SYK miniprep bacmid DNA (Nautilus Genomic DNA Mini Kit Cat. #50050, Active Motif) into 100 μl Sf-900II SFM without pen/strep.

2. Dilute 6 μl of CellFECTIN reagent (Cat. #10362-010 Invitrogen Corporation) in into 100 μl Sf-900II SFM without pen/strep.

Combined the 2 solutions together and incubated 30 minutes at room temperature.

Washed the cells once by aspirating old media and adding Sf-900II SFM without pen/strep.

Removed media and add 0.8 ml Sf-900II SFM without pen/strep to each well. Added lipid/DNA to well.

Incubated 5 hours in 27° C. incubator.

Removed media and replaced with 2 ml Sf-900II SFM containing Penicillin/Streptomycin.

Placed in 27° C. incubator.

Day 3, P1 to P2

In a T75 Tissue Culture Flask seeded 6×10E6 SF9 cells in a total volume of 14 ml Sf-900II SFM containing Penicillin/Streptomycin. Allowed to attach for 1 hour.

Using a 5 ml pipet removed supernatant containing infectious P1 SYK Baculovirus particles from the transfected well of the 6 well and transferred directly into T75 Flask.

Placed in 27° C. incubator.

Day 10, P2 to P3

On Day 10 Harvested SYK Baculovirus supernatant and cells by vigorously pipeting the media to remove the cells from the flask wall.

Pipeted the media and cells into a 15 ml sterile conical tube and centrifuged the tube at @2000 rpm at room temperature for 5 minutes. Saved supernatant (P2).

Cells were analyzed for protein expression by western blot.

P3 infection

Seeded SF21 cells in a 500 ml suspension flask at 2×10E6 cells per ml. In a total volume of 100 ml.

Added infectious SYK supernatant (14 ml) from P2 expression to suspension flask Incubated at 27° C., shaking at 120-130 rpm.

Expressed protein for 72 hours.

Harvested 1 ml cells and western blot to determine expression

Harvested P3 supernatant by centrifugation 3000 rpm for 15 minutes at room temperature.

Sterile filtered viral supernatant.

SYK Scale Up

Seeded 6 liters of SF21 cells at 2×10E6 cells per ml in 1 liter of cells in 2-liter suspension flasks. Infected cells with 15 ml of P3 SYK baculovirus per liter. Incubated at 27° C., shaking at 120-130 rpm.

Expressed protein for 48 hours. Harvested 1 ml cells from each liter and western blot to determine expression. Remaining cells were collected by centrifugation, and the pellets stored at −80° C.

After thawing at room remperature, cells were lysed in cracking buffer (50 mM Tris-HCl, pH 8.0; 200 mM arginine; 150 mM NaCl; 10% glycerol; 0.1% Igepal 630), and centrifuged to remove cell debris. The soluble fraction was purified over an IMAC column charged with nickel (Pharmacia, Uppsala, Sweden), and eluted under native conditions with a step gradient of 400 mM imidazole in 50 mM Tris pH7.8, 10 mM methionine, 10% glycerol. The SYK protein was then purified by gel filtration using a Superdex 200 preparative grade column equilibrated in GF4 buffer (10 mM HEPES, pH 7.5, 10 mM methionine, 500 mM NaCl, 5 mM DTT, and 10% glycerol). Fractions containing the purified SYK kinase domain were pooled and concentrated to 13.2 mg/ml. The protein obtained was >98% pure as judged by mass spectroscopic analysis. Mass spectroscopic analysis of the purified protein showed that it was not phosphorylated.

### EXAMPLE 8

### Crystallization of Target Protein

Various methods known in the art may be used to produce the native and heavy-atom derivative crystals of the present invention. Methods include, but are not limited to, batch, liquid bridge, dialysis, and vapor diffusion (see, e.g., McPherson, Crystallization of Biological Macromolecules, Cold Spring Harbor Press, New York, 1998; McPherson, Eur. J. Biochem. 189:1-23, 1990; Weber, Adv. Protein Chem. 41:1-36, 1991; Methods in Enzymology 276:13-22, 100-110; 131-143, Academic Press, San Diego, 1997).

Generally, native crystals are grown by dissolving substantially pure target protein polypeptide in an aqueous buffer containing a precipitant at a concentration just below that necessary to precipitate the protein. Examples of precipitants include, but are not limited to, polyethylene glycol, ammonium sulfate, 2-methyl-2,4-pentanediol, sodium citrate, sodium chloride, glycerol, isopropanol, lithium sulfate, sodium acetate, sodium formate, potassium sodium tartrate, ethanol, hexanediol, ethylene glycol, dioxane, t-butanol and combinations thereof. Water is removed by controlled evaporation to produce precipitating conditions, which are maintained until crystal growth ceases.

In one embodiment, native crystals are grown by vapor diffusion in hanging drops or sitting drops (McPherson, Preparation and Analysis of Protein Crystals, John Wiley, New York, 1982; McPherson, Eur. J. Biochem. 189:1-23, 1990). Generally, up to about 25 μL, preferably up to about 5 μl, 3 μl, 2 μl, or 1 μl of substantially pure polypeptide solution is mixed with a volume of reservoir solution. The ratio may vary according to biophysical conditions, for example, the ratio of protein volume: reservoir volume in the drop may be 1:1, giving a precipitant concentration about half that required for crystallization. Those of ordinary skill in the art recognize that the drop and reservoir volumes may be varied within certain biophysical conditions and still allow crystallization. In the sitting drop method, the polypeptide/precipitant solution is allowed to equilibrate in a closed container with a larger aqueous reservoir having a precipitant concentration optimal for producing crystals. In the hanging drop method, the polypeptide solution mixed with reservoir solution is suspended as a droplet underneath, for example, a coverslip, which is sealed onto the top of the reservoir. For both methods, the sealed container is allowed to stand, usually, for example, for up to 2-6 weeks, until crystals grow. It is preferable to check the drop periodically to determine if a crystal has formed. One way of viewing the drop is using, for example, a microscope. One method of checking the drop, for high throughput purposes, includes methods that may be found in, for example, U.S. Utility patent application Ser. No. 10/042,929, filed Oct. 18, 2001, entitled “Apparatus and Method for Identification of Crystals By In-situ X-Ray Diffraction.” Such methods include, for example, using an automated apparatus comprising a crystal growing incubator, an X-ray source adjacent to the crystal growing incubator, where the X-ray source is configured to irradiate the crystalline material grown in the crystal growing incubator, and an X-ray detector configured to detect the presence of the diffracted X-rays from crystalline material grown in the incubator. In more preferred methods, a charge coupled video camera is included in the detector system.

Those having skill in the art will recognize that the above-described crystallization conditions can be varied. Such variations may be used alone or in combination, and may include various volumes of protein solution and reservoir solution known to those of ordinary skill in the art. Other buffer solutions may be used such as Tris, imidazole, or MOPS buffer, so long as the desired pH range is maintained, and the chemical composition of the buffer is compatible with crystal formation.

Heavy-atom derivative crystals can be obtained by soaking native crystals in mother liquor containing salts of heavy metal atoms and can also be obtained from SeMet and/or SeCys mutants, as described above for native crystals.

Mutant proteins may crystallize under slightly different crystallization conditions than wild-type protein, or under very different crystallization conditions, depending on the nature of the mutation, and its location in the protein. For example, a non-conservative mutation may result in alteration of the hydrophilicity of the mutant, which may in turn make the mutant protein either more soluble or less soluble than the wild-type protein. Typically, if a protein becomes more hydrophilic as a result of a mutation, it will be more soluble than the wild-type protein in an aqueous solution and a higher precipitant concentration will be needed to cause it to crystallize. Conversely, if a protein becomes less hydrophilic as a result of a mutation, it will be less soluble in an aqueous solution and a lower precipitant concentration will be needed to cause it to crystallize. If the mutation happens to be in a region of the protein involved in crystal lattice contacts, crystallization conditions may be affected in more unpredictable ways.

### EXAMPLE 8.1

### Crystallization and Structure Determination of SYK Kinase Domain

For crystals of Homo sapiens SYK from which the molecular structure coordinates of the invention are obtained, it has been found that a sitting drop containing 1 μl of SYK polypeptide (13.2 mg/ml) in 10 mM Hepes, pH 7.5, 10% glycerol, 150 mM NaCl, 5 mM DTT, and 10 mM methionine; and 1 μl reservoir solution: 25% (v/v) PEG 3350, and 100 mM Tris (pH 8.5), in a sealed container containing 100 μL reservoir solution, incubated overnight (12 hours) at 25° C. provides diffraction quality crystals. One hour before setting up the trays, 1 mM AMP-PNP and 2 mM MgCl₂ were added to the polypeptide.

Those of ordinary skill in the art recognize that the drop and reservoir volumes may be varied within certain biophysical conditions, up to about 10%, 25%, 40% or 50% greater or less than those stated here, and still allow crystallization.

### EXAMPLE 8.2

### Crystal Diffraction Data Collection

The crystals were individually harvested from their trays and transferred to a cryoprotectant consisting of reservoir solution plus 15% glycerol. After about 2 minutes the crystal was collected and transferred into liquid nitrogen. The crystals were then transferred in liquid nitrogen to the Advanced Photon Source (Argonne National Laboratory).

### EXAMPLE 8.3

### Structure Determination

X-ray diffraction data were indexed and integrated using the program MOSFLM (Collaborative Computational Project, Number 4, Acta. Cryst. D50, 760-63, 1994; www.ccp4.ac.uk/main.html) and then merged using the program SCALA (Collaborative Computational Project, Number 4, Acta. Cryst. D50, 760-63, 1994; www.ccp4.ac.uk/main.html). The subsequent conversion of intensity data to structure factor amplitudes was carried out using the program TRUNCATE (Collaborative Computational Project, Number 4, Acta. Cryst. D50, 760-763, 1994; www.ccp4.ac.uk/main.html). An initial model was obtained by molecular replacement using 1 IEP.pdb as a search model using the program MOLREP. (Collaborative Computational Project, Number 4, Acta. Cryst. D50, 760-63, 1994). The initial protein model was built into the resulting map using the program XTALVIEW/XFIT (McRee, D. E. J. Structural Biology, 125:156-65, 1993; available from CCMS (San Diego Super Computer Center) CCMS-request@sdsc.edu.). This model was refined using the program REFMAC (Collaborative Computational Project, Number 4, Acta. Cryst. D50, 760-63, 1994; www.ccp4.ac.uk/main.html) with interactive refitting carried out using the program XTALVIEW/XFIT (McRee, D. E. J. Structural Biology, 125:156-65, 1993; available from CCMS (San Diego Super Computer Center) CCMS-request@sdsc.edu). The stereochemical quality of the atomic model was monitored using PROCHECK (Laskowski et al., J. Appl. Cryst. 26, 283-91, 1993) and WHATCHECK (Vriend, G., J. Mol. Graph 8:52-56, 1990; Hooft, R. W. W. et al., Nature 381:272, 1996) and the agreement of the model with the x-ray data was analyzed using SFCHECK (Collaborative Computational Project, Number 4, Acta. Cryst. D50, 760-63, 1994); www.ccp4.ac.uk/main.html).

TABLE 3
Data Collection Statistics

| Space group | P 1 21 1 |
|---|---|
| Cell dimensions | a = 38.82 Å |
| | b = 84.28 Å |
| | c = 40.25 Å |
| | α = 90° |
| | β = 99.57° |
| | γ = 90° |
| Wavelength λ | 0.9794 Å |
| Overall Resolution limits | 42.258 Å |
| | 2.1 Å |
| Number of reflections collected | 141328 |
| Number of unique reflections | 20850 |
| Overall Redundancy of data | 7 |
| Overall Completeness of data | 96.6% |
| Completeness of data in last data shell | 97.9% |
| Overall R_{SYM} | 0.088 |
| R_{SYM} in last resolved shell | 0.353 |
| Overall I/sigma(I) | 11.5 |
| I/sigma(I) in last shell | 5.5 |

TABLE 4
Model Refinement Statistics

| | | |
|---|---|---|
| Model | Total number of atoms | 2018 |
| | Number of water molecules | 48 |
| | Temperature factor for all atoms | 40.62 Å² |
| | Matthews coefficient | 2.30 |
| | Corresponding solvent content | 46.05% |
| Refinement | Resolution limits | 42.258 Å |
| | | 2.1 Å |
| | Number of reflections used | 14735 |
| | with I > 1 sigma(I) | 14718 |
| | with I > 3 sigma(I) | 11692 |
| | Completeness | 98.5% |
| | R-factor for all reflections | 0.2452 |
| | Correlation coefficient | 0.9193 |
| | Number of reflections above 2 | 13603 |
| | sigma(F) and resolution from 5.0 | |
| | Å - high resolution limit | |
| | used to calculate Rworking | 12918 |
| | used to calculate Rfree | 685 |
| | R-factor without free reflections | 0.229 |
| | R-factor for free reflections | 0.29 |
| | Error in coordinates estimated by | 0.2845 Å |
| | Luzzati plot | |
| Validation | Phi-Psi core region | 89.9% |
| | Phi-Psi violations | 0 |
| | Residues in disallowed regions: | |
| % bad | Short contact distances | 0.4 |
| contacts | | |
| | RMSD from ideal bond length | 0.011 Å |
| | RMSD from ideal bond angle | 1.21° |

### EXAMPLE 8.4

### Structure Analyses

Atomic superpositions were performed with MOE (available from Chemical Computing Group, Inc., Montreal, Quebec, Canada). Per residue solvent accessible surface calculations were done with GRASP (Nicholls et al., “Protein folding and association: insights from the interfacial and thermodynamic properties of hydrocarbons,” Proteins, 11:281-96, 1991). The electrostatic surface was calculated using a probe radius of 1.4 Å.

### EXAMPLE 9

### Crystals of Biological Target Molecule/Compound Interactions

Crystals of complexes of a target protein and a core fragment or compound of the invention may be obtained by a variety of ways known to those of ordinary skill in the art (see, e.g., McPherson, Crystallization of Biological Macromolecules, Cold Spring Harbor Press, New York, 1998; McPherson, Eur. J. Biochem. 189:1-23, 1990; Weber, Adv. Protein Chem. 41:1-36, 1991; Methods in Enzymology 276:13-22, 100-110; 131-143, Academic Press, San Diego, 1997). In one example, the target protein may be incubated in the presence of the compound, or a mixture of compounds, prior to setting up the crystallization trays. In another example, a crystal comprising a target protein may be soaked in a solution comprising the compound, or a mixture of compounds. In this soaking method, it is desirable to have a target protein that has an empty and available binding site. This may be obtained by, for example, obtaining crystals of the protein alone, in the absence of ligand, or, for example, obtaining crystals of the protein in the presence of a ligand that is then soaked out of the binding site either before, or at the same time as, the crystal is soaked in the presence of the compound. The compound, or mixture of compounds, may be dissolved in a solvent that does not dissolve the crystal, or cause detrimental conformational changes, such as, for example, DMSO. The biological target protein may be combined with test core fragments or compounds singly or in groups. For example, a protein or protein crystal may be incubated with a mixture of test core fragments or test core compounds, such as, for example, as discussed in Nienaber et al., U.S. Pat. No. 6,297,021.

### EXAMPLE 9.1

### Soaking of a SYK Crystal in Presence of an Inhibitor Compound

Purified SYK KD protein was obtained as in Example 7. Crystallization conditions were as in Example 8, with the exception that the crystals were obtained using a reservoir solution of 100 mM Hepes (pH 7.0), and 10% PEG 6K, (v/v) incubated for seven days at 4° C. Crystals were harvested and, before data collection, crystals were soaked in 50 microliters of mother liquor after adding 0.5 microliters of a 0.01 mg/ml solution of staurosporin in dimethylsulfoxide.

The crystal data was collected as in Example 8, and the structure determination was essentially as in Example 8, with the exception that the Example 8 model was used as the reference model for molecular replacement.

### EXAMPLE 10

### Characterization of Crystals

The dimensions of a unit cell of a crystal are defined by six numbers, the lengths of three unique edges, a, b, and c, and three unique angles α, β, and γ. The type of unit cell that comprises a crystal is dependent on the values of these variables, as discussed above.

When a crystal is exposed to an X-ray beam, the electrons of the molecules in the crystal diffract the beam such that there is a sphere of diffracted X-rays around the crystal. The angle at which diffracted beams emerge from the crystal can be computed by treating diffraction as if it were reflections from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the 1 index gives the number of parts into which the c edge of the unit cell is cut by the set of hkl planes. Thus, for example, the 235 planes cut the a edge of each unit cell into halves, the b edge of each unit cell into thirds, and the c edge of each unit cell into fifths. Planes that are parallel to the bc face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, may be recorded of a still crystal (not rotated) to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections are recorded on the detector, resulting in a diffraction pattern.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume (Matthews, J. Mol. Biol. 33(2):491-97, 1968).

Collection of Data and Determination of Structure Solutions

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as a Rigaku RU-200, a micro source or mini-source, a sealed-beam source, or a beam line at a synchrotron light source, such as the Advanced Photon Source at Argonne National Laboratory. For use at a rotating anode X-ray generator, preferred anomalous scatterers include, but are not limited to I, Cl, S, and Br. For use at a synchrotron light source, preferred anomalous scatterers include, but are not limited to Br, I, Cl, S, and Se. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

One of the biggest problems in data collection, particularly from macromolecular crystals having a high solvent content, is the rapid degradation of the crystal in the X-ray beam. In order to slow the degradation, data is often collected from a crystal at liquid nitrogen temperatures. In order for a crystal to survive the initial exposure to liquid nitrogen, the formation of ice within the crystal may be prevented by the use of a cryoprotectant. Suitable cryoprotectants include, but are not limited to, low molecular weight polyethylene glycols, ethylene glycol, sucrose, glycerol, xylitol, and combinations thereof. Crystals may be soaked in a solution comprising the one or more cryoprotectants prior to exposure to liquid nitrogen, or the one or more cryoprotectants may be added to the crystallization solution. Data collection at liquid nitrogen temperatures may allow the collection of an entire dataset from one crystal.

Data collection may be performed at optimal energy levels that, as one of ordinary skill in the art is aware, may be dependent on various factors such as, for example, the type of core fragment or compound in the crystal and the particular beamline. Each beamline is calibrated individually by researchers. In one example, the sector 31 ID beamline at the APS in Argonne, Ill., is used to obtain a calibration for the peak or maximum x-ray absorption of a sample of pure selenomethionine of 12,659.4 +/−0.3 electron volts. For crystals comprising core fragments or compounds comprising a covalently-linked bromine, at the APS, a range of, for example, 13,476 to 13,480 electron volts, for example 13,476 electron volts, 816.6 electron volts higher energy than the energy of maximum absorption of selenomethionine, may be used. Greater x-ray energies may be used, with some dimunition of the signal from the bromine atom.

Once a dataset is collected, the information is used to determine the three-dimensional structure of the molecule in the crystal. This phase information may be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal (Blundell et al., Protein Crystallography, Academic Press, 1976).

Another method of obtaining phase information is by molecular replacement, which is a method of calculating initial phases for a new crystal of a polypeptide whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the molecules comprising the new crystal (Lattman, Methods in Enzymology 115:55-77, 1985; Rossmann, “The Molecular Replacement Method,” Int. Sci. Rev. Ser. No. 13, Gordon & Breach, New York, 1972).

A third method of phase determination is multi-wavelength anomalous diffraction or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, Trans. Am. Crystallogr. Assoc., 21:11, 1985; Hendrickson et al., EMBO J. 9:1665, 1990; and Hendrickson, Science, 254:51-58, 1991).

A fourth method of determining phase information is single wavelength anomalous dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen, et al., Acta Cryst., D56:431-41, 2000.

A fifth method of determining phase information is single isomorphous replacement with anomalous scattering or SIRAS. SIRAS combines isomorphous replacement and anomalous scattering techniques to provide phase information for a crystal of a polypeptide. X-ray diffraction data are collected at a single wavelength, usually from both a native and a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is extracted from both the location of the heavy atoms and from anomalous scattering of the heavy atoms. A detailed discussion of SIRAS analysis can be found in North, Acta Cryst. 18:212-16, 1965; Matthews, Acta Cryst. 20:82-86, 1966; Methods in Enzymology 276:530-37, 1997.

Once phase information is obtained, it is combined with the diffraction data to produce an electron density map, an image of the electron clouds surrounding the atoms that constitute the molecules in the unit cell. The higher the resolution of the data, the more distinguishable the features of the electron density map, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically reasonable conformation that fits the map precisely.

After a model is generated, a structure is refined. Refinement is the process of minimizing the function φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function φ converges on a minimum wherein the model fits the electron density map and is stereochemically and conformationally reasonable. During the last stages of refinement, ordered solvent molecules are added to the structure.

### EXAMPLE 11

### Structure Determination Using Molecular Replacement

X-ray diffraction data are indexed and integrated using the program MOSFLM (Collaborative Computational Project, Number 4, Acta. Cryst. D50, 760-63, 1994; www.ccp4.ac.uk/main) and then merged using the program SCALA (Collaborative Computational Project, Number 4, Acta. Cryst. D50, 760-63, 1994). The subsequent conversion of intensity data to structure factor amplitudes is carried out using the program TRUNCATE (Collaborative Computational Project, Number 4, Acta. Cryst. D50, 760-763, 1994). A molecular replacement model from a known structure is positioned in the unit cell of the target protein crystals using EPMR (Kissinger, et al., 1999, Rapid Automated Molecular Replacement by Evolutionary Search, Acta Crystallographica, D55, 484-491, 1999).

This model is refined using the programs REFMAC (Collaborative Computational Project, Number 4, Acta. Cryst. D50, 760-63, 1994) and CNX (Brunger et al. Acta Cryst. D53, 240-55, 2000; Molecular Simulations, Crystallography and NMR Explorer 2000.1) with interactive refitting carried out using the program XTALVIEW/XFIT (McRee, D. E. J. Structural Biology, 125:156-65, 1993; available from CCMS (San Diego Super Computer Center) CCMS-reguest@sdsc.edu). The stereochemical quality of the atomic model is monitored using PROCHECK (Laskowski et al., J. Appl. Cryst. 26, 283-91, 1993) and WHATCHECK (Vriend, G., J. Mol. Graph 8:52-56, 1990; Hooft, R. W. W. et al., Nature 381:272, 1996) and the agreement of the model with the x-ray data is analyzed using SFCHECK (Collaborative Computational Project, Number 4, Acta. Cryst. D50, 760-63, 1994)).

One method that may be employed for this purpose is molecular replacement. In this method, the unknown crystal structure, such as a target protein complex containing a core fragment or compound of the invention, may be determined using phase information from the target protein structure coordinates. This method may provide an accurate three-dimensional structure for the unknown protein in the new crystal more quickly and efficiently than attempting to determine such information ab initio. Potential sites for modification within the various binding sites of the protein may thus be identified for additional interactions with a core fragment or compound of the invention upon derivation thereof according to the instant disclosure. This information provides an additional tool for determining the most efficient binding interactions, for example, increased hydrophobic interactions, between target protein and a chemical group or compound.

If an unknown crystal form has the same space group as and similar cell dimensions to the known target protein crystal form, then the phases derived from the known crystal form can be directly applied to the unknown crystal form, and in turn, an electron density map for the unknown crystal form can be calculated. Difference electron density maps can then be used to examine the differences between the unknown crystal form and the known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. For example, if the unknown crystal form is of a target protein complex, then a difference electron density map between this map and the map derived from the native, uncomplexed crystal will ideally show only the electron density of the ligand. Similarly, if amino acid side chains have different conformations in the two crystal forms, then those differences will be highlighted by peaks (positive electron density) and valleys (negative electron density) in the difference electron density map, making the differences between the two crystal forms easy to detect. However, if the space groups and/or cell dimensions of the two crystal forms are different, then this approach will not work and molecular replacement must be used in order to derive phases for the unknown crystal form.

All of the complexes referred to above may be studied using well-known X-ray diffraction techniques and may be refined against data extending from about 500 Å to at least 3.0 Å and preferably 1.5 Å , until the refinement has converged to limits accepted by those skilled in the art, such as, but not limited to, R=0.2, Rfree=0.25. This may be determined using computer software, such as X-PLOR, CNX, or REFMAC (part of the CCP4 suite; Collaborative Computational Project, Number 4, “The CCP4 Suite: Programs for Protein Crystallography,” Acta Cryst. D50, 760-63, 1994). See, e.g., Blundell et al., Protein Crystallography, Academic Press; Methods in Enzymology, Vols. 114 & 115, 1976; Wyckoff et al., eds., Academic Press, 1985; Methods in Enzymology, Vols. 276 and 277 (Carter & Sweet, eds., Academic Press 1997); “Application of Maximum Likelihood Refinement” G. Murshudov, A. Vagin and E. Dodson, (1996) in the Refinement of Protein Structures, Proceedings of Daresbury Study Weekend; G. N. Murshudov, A. A.Vaginand E. J. Dodson, Acta Cryst. D53, 240-55, 1997; G. N. Murshudov, A. Lebedev, A. A. Vagin, K. S. Wilson and E. J. Dodson, Acta Cryst. Section D55, 247-55, 1999. See, e.g., Blundell et al., Protein Crystallography, Academic Press; Methods in Enzymology, Vols. 114 & 115, 1976; Wyckoff et al., eds., Academic Press, Methods in Enzymology, Vols. 276 and 277, 1985 (Carter & Sweet, eds., Academic Press 1997). This information may thus be used to optimize target protein binding core fragments and compounds of the invention, and more importantly, to design and synthesize additional core fragments and compounds.

### EXAMPLE 12

### X-Ray Structural Analysis of Protein-Ligand Complexes

X-ray diffraction data are indexed and integrated using the program MOSFLM (Collaborative Computational Project, Number 4, Acta. Cryst. D50, 760-63, 1994)and then merged using the program SCALA (Collaborative Computational Project, Number 4, Acta. Cryst. D50, 760-63, 1994). The subsequent conversion of intensity data to structure factor amplitudes is carried out using the program TRUNCATE (Collaborative Computational Project, Number 4, Acta. Cryst. D50, 760-763, 1994). The electron density map is calculated using the coordinates of the protein determined previously by one of the methods described above and using the programs SFALL and FFT (Collaborative Computational Project, Number 4, Acta. Cryst. D50, 760-63, 1994). For ligands containing anomalous scatterers, the anomalous difference map is calculated using the program SFALL and FFT (Collaborative Computational Project, Number 4, Acta. Cryst. D50, 760-63, 1994).

The ligand, such as a core fragment or compound of the invention, is built into the map and adjustments made to the protein model using the program XTALVIEW/XFIT (McRee, D. E. J. Structural Biology, 125:156-65, 1993, available from CCMS (San Diego Super Computer Center) CCMS-request@sdsc.edu.). This model of the protein-ligand complex is refined using the program REFMAC (Collaborative Computational Project, Number 4, Acta. Cryst. D50, 760-63, 1994; www.ccp4.ac.uk/main) or CNX (Brunger et al. Acta Cryst. D53, 240-55, 2000; Molecular Simulations, Crystallography and NMR Explorer 2000.1) with interactive refitting carried out using the program XTALVIEW/XFIT (McRee, D. E. J. Structural Biology, 125:156-65, 1993; available from CCMS (San Diego Super Computer Center) CCMS-request@sdsc.edu). The stereochemical quality of the atomic model is monitored using PROCHECK (Laskowski et al., J. Appl. Cryst. 26, 283-91, 1993) and WHATCHECK (Vriend, G., J. Mol. Graph 8:52-56, 1990; Hooft, R. W. W. et al., Nature 381:272, 1996) and the agreement of the model with the x-ray data is analyzed using SFCHECK (Collaborative Computational Project, Number 4, Acta. Cryst. D50, 760-63, 1994)).

### EXAMPLE 13

### Formulation and Administration

Pharmaceutical compositions comprising a compound or core fragment of the invention are provided by the invention. They may be, for example, target protein modulators such as, for example, inhibitors, which are useful, for example, as antimicrobial agents, as antiviral agents, for modulating protein kinase activity, treatment of conditions mediated by human signal-transduction kinase activity such cancer and neurodegenerative disorders, as well as disease associated with aberrant cytoskeletal rearrangement, neuronal cell differentiation, and cell cycle progression. Pharmaceutical preparations of the present invention are also useful in PET studies, using isotope derivatives of the compounds, such as, for example, ¹⁹F, 11O, and ¹²C.

While the compounds and core fragments will typically be used in therapy for human patients, they may also be used in veterinary medicine to treat similar or identical diseases, and may also be used as agents for agricultural use, for example, as herbicides, fungicides, or pesticides. Pharmaceutical compositions containing target protein affecters may also be used to modify the activity of homologs of target protein. The compounds of the present invention include geometric and optical isomers.

In therapeutic and/or diagnostic applications, the compounds and core fragments of the invention can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds according to the invention are effective over a wide dosage range. For example, in the treatment of adult humans, dosages from 0.01 to 1000 mg, preferably from 0.5 to 100 mg, and more preferably from 1 to 50 mg per day, more preferably from 5 to 40 mg per day may be used. A most preferable dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts of the compounds and core fragments are generally well known to those of ordinary skill in the art and may include, by way of example but not limitation, acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts may include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, the compounds and core fragments as agent(s) may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the invention may be formulated in aqueous solutions, for example, in physiologically compatible buffers such as Hank's solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active agent(s) are contained in an effective amount to achieve its intended purpose. Determination of the effective amounts is well within the capability of those skilled in the art, especially in light of the detailed disclosure provided herein.

In addition to the active agent(s), these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active agent(s) with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross- linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active agent(s) in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

The present invention is not to be limited in scope by the exemplified embodiments, which are intended as illustrations of single aspects of the invention. Indeed, it will be understood that the invention is capable of further modifications based on the foregoing description and accompanying drawings. This application is intended to cover any variations, uses, or adaptations of the invention following, in general, the principles of the invention and including such departures from the present disclosure, as come within known or customary practice within the art to which the invention pertains and as may be applied to the essential features hereinbefore set forth. References cited throughout this application are examples of the level of skill in the art and are hereby incorporated by reference herein in their entirety, whether previously specifically incorporated or not.

We claim:

1. A core fragment library comprising a plurality of core fragments, wherein said core fragments have the formula 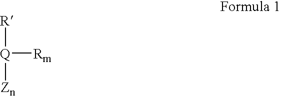
wherein
Z is a handle capable of anomalous dispersion;
Q is a central core;
Q may be the same or different on each compound;
Each R is, independently, H or a handle;
Each R′ is, independently, h or a handle;
n is an integer 0 or greater;
m is an integer 0 or greater; and
(m+n) cannot be greater than the number of available bonds on q:

2. A mixture comprising a biological target molecule and a plurality of core fragments of the library of claim 1.

3. A mixture comprising a biological target molecule and a library of claim 1.

4. A compound library comprising a plurality of compounds, wherein said compounds have the formula 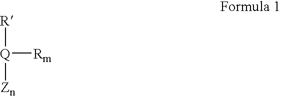
wherein
Z is a handle capable of anomalous dispersion;
Q is a central core, and for each compound, Q is the same;
Each R is, independently, H or a handle;
Each R′ is, independently, H or a derived substituent;
n is an integer 0 or greater; m is an integer 0 or greater; and
(m+n) cannot be greater than the number of available bonds on Q;
with the provisos that
for the majority of compounds in the library, the same R groups are at the same position on Q;
for the majority of compounds in the library, R′ is at the same position on Q; and for the majority of compounds in the library, each n is the same.

5. A mixture comprising a biological target molecule and a compound of the library of claim 4.

6. A method of preparing the mixture of claim 5, comprising
a) Obtaining said library; and
b) Preparing a mixture of a compound of said library and a biological target molecule.

7. Processor executable instructions on one or more computer readable storage devices wherein said instructions cause representation and/or manipulation, via a computer output device, of a core fragment library according to claim 1.

8. Processor executable instructions on one or more computer readable storage devices wherein said instructions cause representation and/or manipulation, via a computer output device, of a core fragment library according to claim 4.

9. A core fragment library comprising a plurality of core fragments wherein each of said core fragments comprises:
a) two or more handles; and
b) less than 17 non-hydrogen atoms.

10. The library of claim 9, wherein at least one of said core fragments comprises at least one single or fused ring system.

11. The library of claim 9, wherein at least one of said core fragments comprises at least one heteroatom on at least one ring.

12. The library of claim 9, wherein at least one of said core fragments comprises at least one hetero atom in the central core.

13. The library of claim 9, wherein,
a) at least 50% of the core fragments have less than four hydrogen bond donors;
b) at least 50% of the core fragments have less than four hydrogen bond acceptors; and
c) at least 50% of the core fragments have a calculated LogP value of less than 4.

14. Processor executable instructions on one or more computer readable storage devices wherein said instructions cause representation and/or manipulation, via a computer output device, of a core fragment library according to claim 9.

15. A core fragment library comprising a plurality of core fragments wherein
a) each of said core fragments comprises two or more handles;
b) at least 50% of the core fragments have less than four hydrogen bond donors;
c) at least 50% of the core fragments have less than four hydrogen bond acceptors; and
d) at least 50% of the core fragments have a calculated LogP value of less than 4.

16. Processor executable instructions on one or more computer readable storage devices wherein said instructions cause representation and/or manipulation, via a computer output device, of a core fragment library according to claim 15.

17. A linear compound library comprising a plurality of compounds, wherein each compound comprises
a) the same central core;
b) n handles, wherein said handles are attached at the same positions on each compound; and
c) at least one derived substituent that differs from the derived substituent on another compound of said library;
wherein said derived substituent is derived from one handle and n+1 is an integer and less than or equal to the number of available bonds on the central core.

18. The library of claim 17, wherein said derived substituents on said compounds have been selected using computational methods.

19. The library of claim 18, wherein said derived substituents on said compounds have been selected to have improved biological activity against a biological target molecule.

20. The library of claim 17, wherein said derived substituents have been selected after a screening step, wherein said screening step comprises obtaining the structure of a core fragment in association with a biological target molecule.

21. The library of claim 17, wherein
50% or more of the compounds have a molecular weight of less than about 300 Daltons; and/or
50% or more of the compounds comprise less than about 5 heteroatoms.

22. Processor executable instructions on one or more computer readable storage devices wherein said instructions cause representation and/or manipulation, via a computer output device, of a library according to claim 17.

23. A compound library comprising two or more compound libraries of claim 17.

24. Processor executable instructions on one or more computer readable storage devices wherein said instructions cause representation and/or manipulation, via a computer output device, of a library according to claim 23.

25. A combination of structures for analysis, said combination comprising a library according to claim 1, or a member of said library, and a biological target molecule, wherein said structures comprise member(s) of said library, said target molecule, and combinations thereof.

26. A combination of structures for analysis, said combination comprising a library according to claim 4, or a member of said library, and a biological target molecule, wherein said structures comprise member(s) of said library, said target molecule, and combinations thereof.

27. A combination of structures for analysis, said combination comprising a library according to claim 9, or a member of said library, and a biological target molecule, wherein said structures comprise member(s) of said library, said target molecule, and combinations thereof.

28. A combination of structures for analysis, said combination comprising a library according to claim 15, or a member of said library, and a biological target molecule, wherein said structures comprise member(s) of said library, said target molecule, and combinations thereof.

29. A combination of structures for analysis, said combination comprising a library according to claim 17, or a member of said library, and a biological target molecule, wherein said structures comprise member(s) of said library, said target molecule, and combinations thereof.

30. A combination of structures for analysis, said combination comprising a library according to claim 23, or a member of said library, and a biological target molecule, wherein said structures comprise member(s) of said library, said target molecule, and combinations thereof.

31. A mixture for analysis by x-ray crystallography, said mixture comprising a plurality of core fragments selected from a library according to claim 1 and a biological target molecule.

32. A mixture for analysis by x-ray crystallography, said mixture comprising a plurality of core fragments selected from a library according to claim 9 and a biological target molecule.

33. A mixture for analysis by x-ray crystallography, said mixture comprising a plurality of core fragments selected from a library according to claim 15 and a biological target molecule.

34. A method of designing a lead candidate having activity against a biological target molecule, comprising
a. Obtaining a library of claim 1;
b. Determining the structures of one or more, or at least two, members of said library in association with said biological target molecule; and
c. selecting information from the structure(s) to design at least one lead candidate.

35. The method of claim 34, further comprising the step of determining the structure of said lead candidate in association with said biological target molecule.

36. The method of claim 34, further comprising the step of designing at least one second library of compounds wherein
a) each compound of said second library comprises a central core and two or more handles; and
b) each compound of said second library differs from each other compound of said second library by at least one derived substituent.

37. The method of claim 36, wherein said central core and the central core of said lead candidate are the same.

38. The method of claim 36, further comprising the steps of
Obtaining said second library; and
Determining the structures of one or more, or at least two, compounds of said second library in association with said biological target molecule.

39. The method of claim 34, wherein said biological target molecule is a protein.

40. The method of claim 34, wherein said biological target molecule is a nucleic acid.

41. The method of claim 34, further comprising the steps of
selecting information about said structures to design at least one second library,
wherein said second library is derived from at least one core fragment of said core fragment library; and
comprises compounds having modifications on at least one of the handles on said core fragment.

42. A method of designing a lead candidate having activity against a biological target molecule, comprising
a) Obtaining a mixture of claim 2;
b) Determining the structure of at least one core fragment of said mixture in association with said biological target molecule;
c) Selecting information from the structure to design at least one lead candidate.

43. A method of designing a candidate compound having activity against a second biological target molecule, comprising
a) Obtaining a lead candidate by the method of claim 34;
b) Determining the interaction of said lead candidate with a second biological target molecule; and
c) Designing at least one second library of compounds wherein each compound of said second library comprises a central core found in said lead candidate and modifications on at least one of the handles on said central core.

44. A method of designing a core fragment library for drug discovery, comprising screening or reviewing a list of synthetically accessible or commercially available core fragments, and selecting core fragments for said library wherein each of said core fragments comprises:
a) two or more handles; and
b) less than 17 non-hydrogen atoms.

45. A method of screening for a core fragment for use as a base core fragment for library design, comprising
a ) Obtaining a library of claim 1;
b) Screening said library for members having binding activity against a biological target molecule; and
c) Selecting a core fragment of member(s) with binding activity to use as a base core fragment for library design.

46. A method of screening for a core fragment for use as a base core fragment for library design, comprising
a) Obtaining a library of claim 9;
b) Screening said library for members having binding activity against a biological target molecule; and
c) Selecting a core fragment of member(s) with binding activity to use as a base core fragment for library design.

47. A method of screening for a core fragment for use as a base core fragment for library design, comprising
a) Obtaining a library of claim 15;
b) Screening said library for members having binding activity against a biological target molecule; and
c) Selecting a core fragment of member(s) with binding activity to use as a base core fragment for library design.

48. A method of identifying a lead candidate having biophysical or biochemical activity against a biological target molecule, comprising
a) Obtaining the structure of said biological target molecule bound to a compound, wherein said compound comprises a first handle having anomalous dispersion properties;
b) Synthesizing a lead candidate molecule comprising the step of replacing said handle on said compound with a second substituent comprising a functionalized carbon, nitrogen, oxygen, or sulfur atom;
c) Assaying said lead candidate molecule for biophysical or biochemical activity against said biological target molecule to identify a lead candidate.

49. The method of claim 48, wherein said second substituent comprises a functionalized carbon or nitrogen atom.

50. A method of designing a lead candidate having biophysical or biochemical activity against a biological target molecule, comprising
a) Combining a biological target molecule with a mixture comprising at least two compounds, wherein at least one of said compounds comprises a substituent having anomalous dispersion properties;
b) Identifying a compound bound to said biological target molecule;
c) Synthesizing a lead candidate molecule comprising the step of replacing said anomalous dispersion substituent with a substituent comprising a functionalized carbon, nitrogen, oxygen, or sulfur atom;
d) Assaying said lead candidate molecule for biophysical or biochemical activity against said biological target molecule.

51. A lead candidate obtained by the method of claim 34.

52. A lead candidate obtained by the method of claim 48.

53. A lead candidate obtained by the method of claim 50.

54. A library obtained by the method of claim 44.

55. A library comprising core fragments selected by the method of claim 47.

56. A candidate compound obtained by the method of claim 43.
